(19) United States
(12) Patent Application Publication
(10) Pub. No.: US 2010/0129383 A1
(43) Pub. Date: May 27, 2010

(54) BIFUNCTIONAL FUSION MOLECULES FOR THE DELIVERY OF ANTIGENS TO PROFESSIONAL ANTIGEN-PRESENTING CELLS

(75) Inventors: MAVANUR R. SURESH, EDMONTON (CA); WEN SHANG WANG, SURREY (CA)

Correspondence Address: SMART & BIGGAR;P.O. BOX 2999, STATION D, 900-55 METCALFE STREET, OTTAWA, ON, K1P 5Y6, CA

(73) Assignee: THE GOVERNORS OF THE UNIVERSITY OF ALBERTA, EDMONTON (CA)

(21) Appl. No.: 12/571,872

(22) Filed: Oct. 1, 2009

Related U.S. Application Data
(60) Provisional application No. 61/102,540, filed on Oct. 3, 2008.

(51) Int. Cl. A61K 39/00; C07K 16/00; C12N 5/0784; A61P 37/04

(52) U.S. Cl. 424/178.1; 530/387.3; 530/391.7; 435/325

(57) ABSTRACT

The invention provides a bifunctional fusion molecule comprising: a first functional domain comprising a first immunoglobulin variable region, a second immunoglobulin variable region and a linker for connecting the first and second variable regions; a second functional domain comprising a moiety for binding to an antigenic agent; wherein the first and second functional domains are linked; and wherein the first functional domain specifically binds to a surface molecule of a professional antigen-presenting cell, and uses thereof.

### CROSS-REFERENCE TO RELATED APPLICATIONS

This application claims the benefit of and priority from U.S. Provisional Application No. 61/102,540 filed on Oct. 3, 2008, which is incorporated herein by reference.

### FIELD OF THE INVENTION

The present invention relates generally to bifunctional fusion molecules for the delivery of antigens to professional antigen-presenting cells.

### BACKGROUND OF THE INVENTION

In vivo targeting of the dendritic cell DEC-205 receptor has been shown to be effective in generating immune responses to protect the host against cancer, viral infection and autoimmune disease. Four types of DEC-205 targeting systems have been reported to date, including: a) HB290 scFv coated liposome [van Broekhoven et al. Cancer Res, 64:4357-65, 2004]; b) chemical cross-linking of HB290 with antigen [Mahnke et al. Cancer Res, 65:7007-12, 2005; Bonifaz et al. J Exp Med, 199:815-24, 2004; Bruder et al. Diabetes, 54:3395-401, 2005; Bonifaz et al. J Exp Med, 196:1627-38, 2002]; c) HB290 hybrid antibody [Trumpfheller et al. J Exp Med. 203:607-17, 2006; Bozzacco et al. Proc Natl Acad Sci USA, 104:1289-94, 2007; Hawiger et al. J Exp Med, 194:769-79, 2001]; and d) bsmAb targeting system [Wang et al. J Immunol Methods, 306:80-92, 2005]. There are several limitations with these systems. Currently, the above strategies have only demonstrated delivery of protein or peptide to dendritic cells. However, these delivery systems may not be versatile and flexible for clinical applications, for example, in the delivery of DNA, glycolipids and multiple, different antigens.

Limitations in the scFv coated liposome targeting strategy include instability of the scFv on the liposome surface (scFv could be lost during circulation); liposome instability; scale-up issues; relative complexity in encapsulation process; and only certain classes of antigens may be encapsulated efficiently (hydrophilic antigens constantly leach from liposome). Issues including batch to batch variation (cross-linking and purification) and Fc domain mediated effects are some of the limitations in chemical cross-linking of the whole mAb with antigen. Hybrid antibody or recombinant antigen fusion protein generation using mammalian expression system requires careful monitoring of in cell productivity, and consideration of issues such as post-translational chemical modifications, degradation and aggregation of the final product [Filpula. Biomol Eng, 24:201-15, 2007]. Moreover, a new antibody fusion protein is required for each antigen (protein or peptide) and the process is often time consuming and costly. A bsmAb produced from a quadroma is also not suitable for clinical applications and the limitations include the yield and purity [Wang et al. J Immunol Methods, 306:80-92, 2005]. Drawbacks for these systems may also include dose-limiting toxicity (prolonged circulation time) and immunogenicity against the targeting system that can alter pharmacokinetics (biodistribution and clearance), block receptor-antigen interactions, induce hypersensitivity reactions and injection site reactions [Filpula. Biomol Eng, 24:201-15, 2007].

Accordingly, there remains a need for molecules useful for delivering antigens to antigen presenting cells.

### SUMMARY OF THE INVENTION

In one aspect, the invention relates to a bifunctional fusion molecule comprising: a first functional domain comprising a first immunoglobulin variable region, a second immunoglobulin variable region and a linker for connecting the first and second variable regions; a second functional domain comprising a moiety for binding to an antigenic agent; wherein the first and second functional domains are linked; and wherein the first functional domain specifically binds to a surface molecule of a professional antigen-presenting cell.

In another aspect, the invention relates to an antigen delivery system comprising: a bifunctional fusion molecule comprising: a first functional domain comprising an immunoglobulin heavy chain variable region, an immunoglobulin light chain variable region and a linker for connecting the heavy chain variable region and the light chain variable region; a second functional domain comprising a moiety for binding to an antigenic agent; wherein the first and second functional domains are linked; and wherein the first functional domain binds to a surface molecule of a professional antigen-presenting cell; and an antigenic agent comprising an antigen and a particle; wherein the antigen is conjugated to the particle, and wherein the particle binds to the moiety of the second domain of the bifunctional fusion molecule.

In another aspect, the invention relates to a method of delivering an antigen to a professional antigen-presenting cell, said method comprising: contacting the professional antigen-presenting cell with the bifunctional fusion molecule as described herein; and contacting the bifunctional fusion molecule with a plurality of the antigenic agents as described herein.

In another aspect, the invention relates to a method of modulating an immune response of a subject to an antigen, said method comprising: administering to the subject the bifunctional fusion molecule as described herein, a plurality of the antigenic agents as described herein and optionally, a co-stimulatory molecule.

In one exemplary embodiment, the invention provides single-chain antibody-core-streptavidin bifunctional fusion molecules (bfFp) that can form a complex with any biotinylated antigen with one arm and targets the antigen to the dendritic cells via the DEC-205 receptor with its second paratope. Such a system has several unique properties over the other targeting systems including dendritic cell targeting with a variety of antigens. Almost any antigen may be biotinylated by chemical conjugation (for instance, NHS-LC-Biotin), photoactivation (photobiotin acetate) or incorporated by synthetic strategies. This simple strategy avoids the need of encapsulation, chemical cross-linking and construction of a new hybrid fusion antibody. The bfFp in addition lacks an Fc domain and the E. coli based production is consistent and economical. Faster clearance rate (kidney glomerular filtration cut-off is 70 kDa) and lower immunogenicities are expected due to smaller molecular weight (˜46 kDa) of the vector. If required, bfFp stability and half-life may be increased by polyethylene glycol linkage [Holliger and Hudson. Nat. Biotechnol, 23:1126-36, 2005] or by isolation of the tetrameric form of bfFp. Moreover, such a targeting vehicle may be translated into clinical applications; several bfFp (tetrameric form) have been applied in clinical studies for pretargeting radioimmunotherapy [Zhang et al. Proc Natl Acad Sci USA, 100:1891-5, 2003; Graves et al. Clin Cancer Res, 9:3712-21, 2003].

Other aspects and features of the present invention will become apparent to those of ordinary skill in the art upon review of the following description of specific embodiments of the invention in conjunction with the accompanying tables and figures.

### BRIEF DESCRIPTION OF DRAWINGS

FIG. 1. The construct of the HB290 scFv (A) V_{H}-V_{L}, (B) V_{L}-V_{H}; and bfFp (C) WET5, (D) WET6 and (E) WET7. The genes were cloned in different orientation to maximize the production in an E. coli system. Abbreviations: pelB, bacterial leader sequence; V_{L}, variable domain light chain; V_{H}, variable domain heavy chain; C_{H1-p}, partial constant heavy chain region 1, 15 amino acids linker; C_{L-p}, partial constant light chain region, 15 amino acids linker; G, glycine; S, serine; His₆, six histidine amino acid tag.

FIG. 2. Expression and purification of recombinant proteins. The scFv and bfFp genes were chemically transformed, cultured, induced and the whole-cell bacterial pellets were analyzed by Western blot. (A) Western blot probed with anti-His₆ mAb for analysis of scFv and bfFp expression. Lane 1: HB290 scFv V_{H}-V_{L}; lane 2: scFv V_{L}-V_{H}; lane 3: bfFp WET5, core-streptavidin-V_{H}-V_{L}; lane 4: bfFp WET6, core-streptavidin-V_{L}-V_{H}; lane 5: bfFp WET7, V_{L}-V_{H}-core-streptavidin. WET7 has the highest protein expression level. (B) SDS-PAGE of WET7 before and after IMAC purification. Lane 1: WET7 periplasmic protein; lane 2: IMAC-purified WET7. M: molecular weight marker. The values at the left of each figure denote molecular weights.

FIG. 3. Demonstration of IMAC-purified bfFp bifunctional activity and thermal stability. (A) IMAC purified bfFp was incubated either at 60° C. (lane 3) or 95° C. (lane 4) for 10 min and probed with B-BSA followed by streptavidin-HRPO. Lanes 1 (HB290 scFv V_{H}-V_{L}) and 2 (HB290 scFv V_{L}-V_{H}) are controls. The values at the left of the figure denote molecular weights. The IMAC-purified bfFp bifunctional activity was tested against DC 2.4 cells and B-OVA via ELISA method (B). Competition binding studies of HB290 mAb and bfFp for binding to dendritic cells. The binding of bfFp is confirmed by B-OVA and streptavidin-HRPO. The error bars are the standard deviations.

FIG. 4. Analysis of humoral immune responses to biotinylated antigens in vivo. Groups of five mice were immunized with different antigen combinations along with a PBS control. The specific components, amounts, and the schedule of immunizations are outlined in Table 2. The mice were analyzed individually and the data was pooled. The humoral response, as quantified by serum antibody titres on day 21 post immunization, was measured by ELISA method against the respective antigens listed in Table 1B. The method involved coating 10 μg of antigen in microtiter plates followed by addition of 1:1000 diluted serum antibody, and detection using GAM-HRPO. The ELISA measurements were done in quadruplicate for each mouse. The mean ELISA values obtained for each individual mouse were further averaged. The error bars represent the standard deviation in a group of 5 mice.

FIG. 5. Analysis of cell-mediated immune responses based on IFN-γ estimation. Spleen T cells (responder cells) from the groups of mice immunized with different antigens (shown on X-axis, and, for details, see Tables 1B and 2) were isolated and purified using a nylon wool column. The five spleens in each group were pooled, mixed in DMEM prior to nylon wool purification. Stimulator cells prepared from the spleen cells of naïve mice were isolated and treated with 50 μg/ml mitomycin C. Purified responder cells from both immunized and non-immunized mice were aliquoted to a tissue culture plate in quadruplicate with or without stimulator cells. The cells were then incubated with 10 μg of antigen for 3 days at 37° C. in a CO₂ atmosphere. After incubation, the IFN-γ concentration in the supernatant was determined using mouse IFN-γ ELISA Ready-SET-Go kit using the protocol from eBioscience. Each data set is shown following subtraction of the corresponding ELISA values obtained without stimulator cells. The error bars are the standard deviations.

FIG. 6. Analysis of serum reactivity towards biotin, bfFp and core-streptavidin. The mice (5 mice per group) were analyzed individually and the data was pooled. The mouse group is listed on the X-axis. The serum reactivity, as quantified by serum antibody titres on day 21 post immunization, was measured by ELISA method against B-BSA, bfFp and core-streptavidin. The method involved coating of 10 μg antigen in microtiter plates followed by addition of 1:1000 diluted serum antibody, and detection using GAM-HRPO. The ELISA measurements were done in quadruplicate for each mouse. The mean ELISA values obtained for each individual mouse were further averaged. The error bars represent the standard deviation in a group of 5 mice.

FIG. 7. Amino acid sequence of the V_{H} region of HB290 (SEQ ID NO: 17).

FIG. 8. Nucleotide sequence of the V_{H} region of HB290 (SEQ ID NO: 18).

FIG. 9. Amino acid sequence of the V_{L} region of HB290 (SEQ ID NO: 19).

FIG. 10. Nucleotide sequence of the V_{L} region of HB290 (SEQ ID NO: 20).

FIG. 11. Amino acid (SEQ ID NO: 22), coding (SEQ ID NO: 21) and non-coding (SEQ ID NO: 23) nucleotide sequences of the V_{H}-C_{H}1 region of HB290.

FIG. 12. Amino acid (SEQ ID NO: 25), coding (SEQ ID NO: 24) and non-coding (SEQ ID NO: 26) nucleotide acid sequences of the V_{L}-C_{L} region of HB290.

### DETAILED DESCRIPTION

The bifunctional fusion molecules of the present invention may be used to deliver one or more antigens, such as an infectious disease antigen, to a professional antigen-presenting cell, such as a dendritic cell.

The in vivo targeting strategy of the present invention is effective in generating an immune response that protects the host against, for example, which is not meant to be limiting, viral infection, autoimmune disease and transplant rejection. The presentation of antigen may result in immunostimulation or immunoregulation depending on the presence or absence, respectively, of co-stimulation of the professional antigen-presenting cell, for example, with a CD40 agonist (such as an anti-CD40 antibody, CpG, polyl:C and MF59). As a result, the bifunctional fusion molecule of the present invention may be used to target one or more antigens to professional antigen-presenting cells, such as dendritic cells, to stimulate an immune response against a viral or bacterial pathogen; or to induce tolerance by delivery antigen in the absence of a co-stimulatory molecule.

Other co-stimulatory molecules that may mature and/or activate professional antigen presenting cells include, but are not limited to, whole bacteria or bacterial-derived antigens, inflammatory cytokines, or other molecules that can cross-link to a select cell surface receptor such as CD40, and viral products

The bifunctional fusion molecules, antigens, methods and uses described herein relate to a bifunctional fusion molecule that has a first functional domain specific for a cell surface molecule of a professional antigen-presenting cell and a second functional domain comprising a moiety that is specific for a particle, where the particle is not a target of the first functional domain, in combination with an antigenic agent comprising an antigen and a particle that associates with the moiety of the second functional domain of the antibody, to direct antigens to a professional antigen-presenting cell. In one embodiment, the bifunctional fusion molecule may be in monomeric form.

As used herein, a “moiety” may comprise any molecule that can associate specifically with the particle of the antigenic agent.

To develop the bifunctional fusion molecule, general molecular approaches may be used to clone the expressed V_{H} and V_{L} fragments from a hybridoma expressing an antibody of known specificity. These fragments are then joined by a linker and combined with a molecule, such as a core streptavidin fragment, resulting in a bifunctional fusion molecule.

Antibodies (also termed immunoglobulins) are protein molecules produced by lymphocytes that bind with high specificity for its cognate antigen. Typically, antibodies consist of two heavy and two light chains that are covalently linked to each other via disulfide bonds. Each chain comprises variable domains and constant domains. A variable domain comprising three complementary regions (CDRs) is located at the N-terminus of each chain. Together, variable regions of the heavy and light chains determine antigen specificity of the antibody. Single chain variable fragment (scFv) antibodies consists of variable heavy (V_{H}) and variable light (V_{L}) domains linked by a flexible amino acid linker (Bird et al. Science, 242:423-426, 1988; Huston et al. Proc Natl Acad Sci USA, 85:5879-5883, 1988).

scFvs are encoded by a single gene and the resulting scFv may be expressed in microbial systems such as yeast and prokaryotic systems (reviewed in Verma et al. J. Immunol. Methods, 216:165-181, 1998), allowing for rapid selection of specific high affinity molecules, using techniques such as phage display or ribosome display. Due to the absence of a Fc portion, scFv antibodies do not exert toxic effects via antibody-dependent or complement-dependent cell-mediated cytotoxicity. Further, scFv antibodies show good tissue penetration abilities. Due to these advantages, scFv fragments have found broad applications in medicine (reviewed by Huston et al. Int Rev Immunol, 10:195-217, 1993) and have potential in biotechnology (Harris. Trends Biotechnol, 17:290-296, 1999).

scFvs have been successfully used in intracellular applications (Worn et al. Curr Opin Rheumatol, 16:38-42, 2000; Auf der Maur et al. J Biol Chem, 277:45075-85, 2002; Stocks. Drug Discov Today, 15:960-966, 2004). In general, intracellular expression of functional scFvs is limited by their instability, insolubility, and tendency to form aggregates. Thus, in vivo screening systems for scFv antibodies have been developed using a “Quality Control” screen (WO0148017; Auf der Maur et al. FEBS Lett 508:407-412, 2001; Auf der Maur et al. Methods, 34:215-224, 2004), leading to the identification of particularly stable and soluble scFv framework sequences (WO03097697). These frameworks are highly expressed, stable and soluble under natural, oxidizing conditions in the extracellular environment. Accordingly, scFvs are suitable for therapeutic applications.

The immunoglobulins of the present invention may be adapted for use in a recipient. For example, murine antibody fragments may be adapted for use in humans by humanizing the fragments. Humanized antibodies may be produced, without limitation, by merging the DNA that encodes the binding portion of a non-human (e.g. mouse) antibody with human antibody-producing DNA. The extent of the CDRs, the human frameworks to use and the substitution of residues from the rodent mAb into the human framework regions (back mutations) may also be considered in the design of the humanized antibody.

As used herein, “linker” refers, without limitation, to a molecule or a chemical bond that links two domains. In one embodiment, the V_{H} and V_{L} domains are linked by chemical cross-linking. In another embodiment, the linker is a peptide linking V_{H} and V_{L} that permits folding of the antibody fragments, i.e., an appropriate folding of the V_{H} and V_{L} domains and their capacity to be brought together. In addition, the linker permits folding into a monomeric functional unit. When the antibody fragments are assembled in the V_{H} to V_{L} orientation (V_{H}-linker-V_{L}), a linker of 3 to 12 residues may not fold into a functional Fv domain and instead may associate with a second molecule to form a bivalent dimer. Reducing below 3 residues may lead to trimers. Direct ligation of V_{L} to V_{H} may lead to the formation of tetramers. A typical linker of the present invention may have at least 12 and preferably less than 25 amino acids, preferably between 14-18, 14-16, or 15 amino acids.

The first functional domain and the second functional domain of the bifunctional fusion molecule may be linked by a linker as described above.

The first functional domain of the bifunctional fusion molecule comprises, consists or consists essentially of a V_{H} region and a V_{L} region joined by a linker. In one embodiment, the first functional domain comprises a scFv antibody.

The first and second functional domains may be joined in any number of configurations. One configuration of the bifunctional fusion molecule is: (NH₃⁺) V_{H}-linker-V_{L}-second functional domain (COOH). Another configuration of the bifunctional fusion molecule is: (NH₃⁺) V_{L}-linker-V_{H}-second functional domain (COOH). Another configuration of the bifunctional fusion molecule is: (COOH) V_{H}-linker-V_{L}-second functional domain (NH₃⁺). A further configuration of the bifunctional fusion molecule is: (COOH) V_{L}-linker-V_{H}-second functional domain (NH₃⁺). The preferred configuration of the bifunctional fusion molecule is: (NH₃⁺) V_{L}-linker-V_{H}-second functional domain (COON).

The second functional domain comprises a moiety that interacts with a cognate particle on an antigenic agent. The second functional domain may comprise any moiety as long as it is not reactive with the immunoglobulin fragments of the first functional domain. In one embodiment, the moiety of the second functional domain is a protein, for instance a protein such as streptavidin, core streptavidin or avidin. In another embodiment, the moiety of the second functional domain is an antibody that binds biotin.

Streptavidin is a 53 kDa tetrameric protein purified from the bacterium Streptomyces avidinii. It finds wide use in molecular biology through its extraordinarily strong affinity for the vitamin biotin; the dissociation constant (Kd) of the biotin-streptavidin complex is on the order of ˜10-15 mol/L, ranking among one of the strongest known non-covalent interactions. Streptavidin mutants may also be engineered in the form of a stable, single-chain dimer, (Asian et al., Proc Natl Acad Sci USA, 102:8507-8512, 2005). Further, streptavidin may be modified, for example, by substitution of charged, aromatic, or large hydrophobic residues on the surface of streptavidin with smaller neutral residues to reduce the protein's immunogenicity (Meyer et al. Protein Science, 10:491-503, 2001). As used herein, “core streptavidin” refers to a streptavidin product that had been reduced to a minimal size that still retains full biotin-binding activity.

The bifunctional fusion molecule may further comprise a tag, for example, a protein tag. Protein tags may find use in protein purification, specific enzymatic modification and chemical modification. Protein tags are known in the art and include, but are not limited to, affinity tags (e.g. poly-Histidine), solubilization tags (e.g. thioredoxin), chromatography tags (e.g. FLAG), epitope tags (e.g. c-myc-tag) and fluorescent tags (e.g. GFP). In some instances, these tags are removable by chemical agents or by enzymatic means.

The first functional domain of the bifunctional fusion molecule targets a cell surface molecule of a professional antigen-presenting cell.

As used herein, a “surface molecule” refers to any molecule that associated with the plasma membrane of a professional antigen-presenting cell. Preferably, the surface molecule is present on the extracellular side of the plasma membrane. Exemplary surface molecules include, but are not limited to, receptors, cell adhesion molecules, cellular transporters and other molecules displayed on the cell surface.

The term “cell” or “cells” refers to a single cell, as well as a plurality of cells, a culture of cells, a growth of cells, a population of cells or a cell line, and may be in vitro or in vivo, where context permits, unless otherwise specified. In vitro cells include ex vivo cells explanted from a subject. Similarly, reference to “cells” also includes reference to a single cell where context permits, unless otherwise specified.

The adaptive immune system has evolved to recognize and respond to a vast number of diverse antigens based on a repertoire of lymphocytes of unique antigen binding specificities. Lymphocytes generally work in tandem with professional antigen-presenting cells which capture antigen and present them to the lymphocytes, resulting in lymphocyte activation or inactivation, depending on the presence or absence of co-stimulatory signals, respectively. Most cells in the body can present antigen to CD8+ T cells via MHC class I molecules however, as used herein, “professional antigen-presenting cells” (or “APCs”) refers generally to those specialized cells that can prime T cells. Professional antigen-presenting cells, in general, express MHC class II as well as MHC class I molecules, and can stimulate CD4+ (“helper”) cells as well as CD8+ (“cytotoxic”) T cells.

There are three main types of professional antigen-presenting cell: 1) Dendritic cells; 2) Macrophages; and 3) B-cells.

Dendritic cells are the most specialized and potent professional antigen-presenting cells in the immune system and play a critical role in innate and adaptive immune responses, especially in priming and activating T cell and B cell immunity. Dendritic cells may originate from the myeloid (including Langerhan cells) or lymphoid (or plasmacytoid) lineage. The term includes dendritic cells at any stage of development, including, but not limited to, immature dendritic cells and activated dendritic cells.

Dendritic cells are motile cells that ingest antigens and present them to B and T lymphocytes. Dendritic cells also play critical roles in the induction of central and peripheral immunological tolerance, the regulation of the types of T cell immune responses, and functions in innate immunity against microbes (reviewed in Sato and Fujita. Allergology International, 56:183-191, 2007; Banchereau and Steinman. Nature, 392:245-252, 1998; Diebold. Immunology and Cell Biology, 86:389-397, 2008).

During maturation, dendritic cells change the uptake, processing and presentation of material from their environment (Mellman and Steinman. Cell, 106:255-258, 2001). Immature dendritic cells constantly ingest material from their environment, but are not efficient in antigen presentation. Once the cells are activated and undergo maturation, processing and presentation of antigens from the ingested material is induced, resulting in increase levels of major histocompatibility complex (MHC) class II molecules at the cell surface (Inaba et al. J Exp Med, 191:927-936, 2000).

Dendritic cells utilize surface receptors (Gb3/CD77, CD40, β2 integrins, Fc receptors, and C-type lectin receptors) to internalize, process and present antigens to MHC class I and MHC class II pathways (Tacken et al. Immunobiology, 211:599-608, 2006). Exemplary dendritic cell surface receptors that may be used for the targeting of antigens to dendritic cells are listed in Table 1.1.

Table 1.1 also shows the expression pattern of the receptors and also indicates whether antigen targeting to the receptor required co-stimulation for induction of immune responses. In summary, antigen targeting to Gb3, LOX-I and CD40 receptors does not require co-stimulation for induction of immune responses. DC-SIGN expression is restricted to dendritic cells and macrophages; whereas other receptors are distributed on a variety of cells. DEC-205 targeting strategies appear to be extensively studied and applied for a variety of applications.

DEC-205 (CD205) is a C-type lectin receptor that is present in both immature and mature dendritic cells in lymphoid tissues, lymph nodes and spleen (Inaba et al. Cell Immunol, 163:148-56, 1995; den Haan et al. J Exp Med, 192:1685-96, 2000; Witmer-Pack et al. Cell Immunol, 163:157-62, 1995; Granelli-Piperno et al. J Immunol, 175:4265-73, 2005; Pack et al. Immunology, 123:438-46, 2008). DEC-205 is also expressed at low levels on macrophages and at moderate levels by B cells and is up-regulated during the pre-B cell to B cell transition (Inaba et al. Cell Immunol, 163:145, 1995). DEC-205 behaves as an antigen uptake/processing receptor for dendritic cells (Kato et al. International Immunology, 18:857-869, 2006). In the presence of co-stimulatory molecules, DEC-205 targeting of antigens by an antibody is an efficient strategy to protect the host against tumor growth (van Broekhoven et al. Cancer Res, 64:4357-65, 2004), rejection of existing tumor (Mahnke et al. Cancer Res, 65:7007-12, 2005), airway challenge of virus (Trumpfheller et al. J Exp Med. 203:607-17, 2006), and to enhance resistance to an established rapidly growing tumor, as well as to viral infection (Bonifaz et al. J Exp Med, 199:815-24, 2004).

DEC-205 targeting of a single protein enables cross-presentation of several peptides more efficiently than CD206 and CD209 (Bozzacco et al. Proc Natl Acad Sci USA, 104:1289-94, 2007) and induces stronger T cell immunity at much lower doses of protein antigen, plasmid DNA or recombinant adenovirus (Trumpfheller at al. J Exp Med. 203:607-17, 2006). In the absence of co-stimulation, DEC-205 targeting of antigen can be used to suppress the development of autoimmunity, such as type 1 diabetes (Bruder et al. Diabetes, 54:3395-401, 2005). Similarly, other studies have demonstrated that DEC-205 targeting, depending on the presence or absence of an additional activation signal (i.e., CD40 agonist, such as an anti-CD40 antibody), may result in either immunostimulatory or immunoregulatory effects, respectively (Bonifaz at al. J Exp Med, 196:1627, 2002; Hawiger et al. J Exp Med, 194:769, 2001; Hawiger et al. Immunity, 20:695, 2004; Bonifaz et al. J Exp Med, 199:815, 2004).

Other dendritic cell surface molecules that may be used in the context of the invention may be identified by methods known in the art. For example, a typical method for identifying other suitable dendritic cells surface molecules as a target comprise first directing a molecule, such as a ligand or an antigen, towards the professional antigen-presenting cell surface molecule. Flow cytometry may be performed on those cells to identify upregulation or downregulation of MHC, CD and/or other cell surface molecules. DNA microarray, 2D SDS-PAGE, cytokine monitoring, cells proliferation study, in vivo fluorescence imaging, in vivo immune system study and animal challenge study may be subsequently used to identify the cell surface molecule.

In one embodiment, the first functional domain of the bifunctional fusion molecule is directed towards DEC-205.

In this regard, the V_{H} region of the bifunctional fusion molecule may comprise, consist of, or consist essentially of an amino acid sequence that has at least 70%, at least 75%, at least 80%, at least 83%, at least 85%, at least 87%, at least 89%, at least 91%, at least 93%, at least 95%, at least 97%, at least 99% or has 100% sequence identity to SEQ ID NO: 17 (amino acid sequence of V_{H}), wherein the V_{H} in association with V_{L} have specificity towards DEC-205.

In one embodiment, the V_{H} region of the bifunctional fusion molecule is encoded by a nucleic acid sequence that has at least 70%, at least 75%, at least 80%, at least 83%, at least 85%, at least 87%, at least 89%, at least 91%, at least 93%, at least 95%, at least 97%, at least 99% or has 100% sequence identity to SEQ ID NO: 18 (nucleic acid sequence of V_{H}), wherein the V_{H} in association with V_{L} have specificity towards DEC-205.

The V_{L} region of the bifunctional fusion molecule may comprise, consist of, or consist essentially of an amino acid sequence that has at least 70%, at least 75%, at least 80%, at least 83%, at least 85%, at least 87%, at least 89%, at least 91%, at least 93%, at least 95%, at least 97%, at least 99% or has 100% sequence identity to SEQ ID NO: 19 (amino acid sequence of V_{L}), wherein the V_{L} in association with V_{H} have specificity towards DEC-205.

In one embodiment, the V_{L} region of the bifunctional fusion molecule is encoded by a nucleic acid sequence that has at least 70%, at least 75%, at least 80%, at least 83%, at least 85%, at least 87%, at least 89%, at least 91%, at least 93%, at least 95%, at least 97%, at least 99% or has 100% sequence identity to SEQ ID NO: 20 (nucleic acid sequence of V_{L}), wherein the V_{L} in association with V_{H} have specificity towards DEC-205.

Fragments of V_{H} and V_{L} regions that retain the binding specificity of the full length V_{H} and V_{L} regions may be used. Such fragments may be e.g. at least 10, 20, 30, 40, 50, 100 or 200 amino acids in length.

As used herein, “% sequence identity” is determined by comparing two optimally aligned sequences over a comparison window, where the fragment of the polypeptide or polynucleotide sequence in the comparison window may comprise additions or deletions (e.g., gaps or overhangs) as compared to the reference sequence (which does not comprise additions or deletions) for optimal alignment of the two sequences. The percentage is calculated by determining the number of positions at which the identical amino acid residue or nucleic acid residue occurs in both sequences to yield the number of matched positions, dividing the number of matched positions by the total number of positions in the comparison window and multiplying the result by 100 to provide the percentage of sequence identity. Algorithms to align sequences are known in the art. Exemplary algorithms include, but are not limited to, the local homology algorithm of Smith and Waterman (Add APL Math, 2:482, 1981); the homology alignment algorithm of Needleman and Wunsch (J Mol Biol, 48:443, 1970); the search for similarity method of Pearson and Lipman (Proc Natl Acad Sci USA, 85:2444, 1988); and computerized implementations of these algorithms (GAP, BESTFIT, BLAST, PASTA, and TFASTA in the Wisconsin Genetics Software Package, Genetics Computer Group (GCG), 575 Science Dr., Madison, Wis.). In one embodiment, two sequences may be aligned using the “Blast 2 Sequences” tool at the NCBI website at default settings (Tatusova and Madden. FEMS Microbiol Lett, 174: 247-250, 1999). Alternatively, amino acid sequences or nucleic acids sequences may be aligned by human inspection.

As used herein, “consists essentially of” or “consisting essentially of” means that the bifunctional fusion molecule may include additional amino acid residues, including within the amino acid sequence or at one or both ends of the amino acid sequence, but that the additional residues do not materially affect the function of the bifunctional fusion molecule.

In this regard, the V_{H} region may include residues from the constant region of the heavy chain (see SEQ ID NO: 21 and SEQ ID NO: 22) and/or the V_{L} regions may include residues from the constant region of the light chain (see SEQ ID NO: 24 and SEQ ID NO: 25).

The bifunctional fusion molecule associates with an antigenic agent through interaction at the second functional domain. An antigenic agent comprises an antigen and a particle, where the particle binds the moiety of the second functional domain of the bifunctional fusion molecule. In one embodiment the antigen is linked to the particle. Further, the antigen and particle of the antigenic agent may be linked by chemical cross-linking or by a linker.

Antigens may comprise, without limitation, proteins, peptides, nucleic acids and/or glycolipids. As used herein, an “antigen” refers to molecules that react with antibodies, B-cell receptors and/or T-cell receptors. Some antigens do not by themselves elicit an immune response.

As used herein, the terms “peptide”, “oligopeptide”, “polypeptide” and “protein” may be used interchangeably. Polypeptides may comprise non-natural amino acids and may be joined to linker elements known to the skilled person. Polypeptides may also comprise modifications to the amino acid side chains or the backbone structure, such modifications may occur during polypeptide synthesis or processing or following treatment with isolated modifying enzymes. Further, polypeptides may be monomeric or multimeric, and may include derivatives, variants, fragments, analogs or homologs thereof.

Polypeptides may comprise a contiguous span of at least 5, at least 10, at least 25, at least 50, at least 100, at least 250, at least 500, at least 1000, at least 1500, or at least 2500 consecutive amino acids and may retain the desired activity and/or structure of the full length polypeptide.

The term “protein” may also refer to a full length or essentially the full length product encoded by a gene. As used herein, “essentially” means that a protein may include or lack one or more amino acid residues, but that the additional or missing amino acid residues do not materially affect the function and/or structure of the protein.

As used herein, “nucleotide sequence”, “nucleic acid” or “nucleic acid molecule” refers to a polymer of DNA or RNA which can be single or double stranded and optionally containing synthetic, non-natural or altered nucleotide bases capable of incorporation into DNA or RNA polymers. “Nucleic acids”, “Nucleic acid sequences” or “Nucleic acid molecules” may encompass genes, cDNA, DNA and RNA encoded by a gene. Nucleic acids or nucleic acid sequences may comprise at least 3, at least 10, at least 100, at least 1000, at least 5000, or at least 10000 nucleotides or base pairs.

As used herein, “glycolipid” refers, without limitation, to any compound containing one or more monosaccharide residues bound by a glycosidic linkage to a hydrophobic moiety including, but not limited to, an acylglycerol, a sphingoid, a ceramide (N-acylsphingoid) or a prenyl phosphate.

The antigens of the invention include, without limitation, those derived from cancer, autoimmune diseases and infectious disease agents such as viruses and bacteria.

Exemplary infectious viral and/or bacterial antigens include proteins derived from, and nucleic acids encoding proteins derived from bacteria and viruses including, but not limited to, avian influenza, ebola, Bacillus anthracis (anthrax), Severe acute respiratory syndrome-coronavirus (SARS-CoV), Western Equine Encephalitis Virus (WEEV), poliovirus, human rhinovirus, hepatitis A virus, human immunodeficiency virus, human influenza, human papillomavirus, herpes simplex virus, picornaviruses such as foot-and-mouth disease virus, Dengue and West Nile viruses, Yersinia pestis, and respiratory syncytial virus.

Exemplary cancer disease associated and/or specific antigens include, but are not limited to, MUC-1 of breast cancer, GM2 and GM3 of myeloma cancer.

Exemplary antigens associated with autoimmune disease include, but are not limited to, transglutaminase in celiac disease, muscle actin in autoimmune hepatitis, Bullous Pemphigoid antigen 1 and 2 in bullous pemphigoid, basement membrane collagen Type IV protein in Goodpasture's syndrome, ganglioside in Guillain-Barre syndrome, myelin basic protein in multiple sclerosis, desmogein 3 in Pemphigus Vulgaris, p62/sp100/mitochondrial(M2) in primary biliary cirrhosis, rheumatoid factor in rheumatoid arthritis, and topoisomerase in Scleroderma.

The antigenic agent further comprises a particle. The particle may be, without limitation, a vitamin, a peptide, a protein, a glycoconjugate or any natural or synthetic conjugate, so long as it associates with the moiety of the second functional domain of the bifunctional fusion molecule but not with the immunoglobulin fragments of the first functional domain. In one embodiment, the antigen may have high affinity specifically for the moiety of the second functional domain of the bifunctional fusion molecule. Pairs of molecules with strong affinity for one another that may be suitable for use in the context of the invention are known in the art and include, but not limited to, streptavidin and biotin, and an antibody and its cognate antigen. The skilled person would appreciate that methods to attach a particle to an antigen are known in the art and that the choice of a suitable method would depend, among other factors, on the nature and/or composition of the antigen and the particle. Typical methods of attaching a particle to an antigen include, but are not limited to, covalent, ionic or electrostatic interactions. In one embodiment, an antigen is attached to a particle by a linker.

As noted above, various configurations of the first and second functional domains are possible. For the purpose of illustration, the following are a number of non-limiting examples of particular combinations of particular bifunctional fusion molecules according to the invention:

1. V_{L}-V_{H}-core streptavidin

2. V_{H}-V_{L}-core streptavidin

3. Core streptavidin-V_{L}-V_{H}

4. Core streptavidin-V_{H}-V_{L}

In Examples 1 to 4, the second functional domain comprises core streptavidin as the moiety which specifically binds a particle (in this instance biotin) attached to the antigen. Alternative suitable moiety/particle pairs may of course be used.

In one embodiment, the moiety of the second functional domain of the bifunctional fusion molecule is core streptavidin or avidin and the particle of the antigenic agent is biotin. Alternatively, the moiety of the second functional domain may be biotin, and the particle of the antigen agent may be core streptavidin or avidin. Biotinylation may be effected by methods known in the art, including, but not limited to, chemical conjugation (for instance, NHS-LC-Biotin), photoactivation (photobiotin acetate) or incorporated by synthetic strategies.

The construction, expression, purification and analysis of the bifunctional fusion molecule and the antigenic agent may be achieved by methods known in the art.

With respect to construction, a skilled person can construct the bifunctional fusion molecule based on known molecular techniques. In one embodiment, the bifunctional fusion molecule may be expressed from a vector encoding V_{H} and V_{L} fragments separated by a linker and fused to a second functional domain. In another embodiment, the first functional domain may comprise variable regions and a linker that were each encoded by separate vectors, expressed and subsequently joined together, for example, by chemical conjugation. In a further embodiment, the V_{H} and V_{L} fragments may be cloned from a hybridoma expressing an antibody of desired specificity. Alternatively, V_{H}, V_{L} and linker fragments may be synthesized chemically separately and then joined together by methods known in the art, or the fragments may be synthesized as a single molecule.

Similarly, the skilled person would know how to construct an antigenic agent comprising an antigen and a particle, depending on the nature of the antigen and that of the particle. The antigen may be chemically synthesized or cloned from a cell expressing the antigen. The antigen and the particle may be expressed and/or produced in tandem or separately.

Methods of expressing the bifunctional fusion molecule and the antigenic agent will depend on their composition. Exemplary methods include microbial, mammalian, plant cell cultures and cell free culture systems known to the skilled person.

The expressed bifunctional fusion molecule and the antigenic agent may be purified with methods and techniques known in the art. Exemplary purification methods include, but are not limited to, affinity chromatography, size exclusion chromatography, Immobilized Metal Chelating Chromatography (IMAC), and agarose/acrylamide gel electrophoresis.

Following purification, the bifunctional fusion molecule and the antigenic agent may be detected and/or assessed for biological activity. Exemplary methods of detecting the bifunctional fusion molecule and the antigenic agent may include, but are not limited to, Western blot analysis, ELISA and gas chromatography. Exemplary methods of assessing the biological function of the bifunctional fusion molecule and the antigenic agent may include, but are not limited to, serum neutralization inhibition assays, and in vivo assays to assess the antibody production as indication of a humoral immune response and IFN-γ as indication of cell mediated immune response.

The bifunctional fusion molecule and the antigenic agent may be used serially, in either order, simultaneously or as part of a treatment strategy. In one embodiment, there is provided a pharmaceutical composition comprising a bifunctional fusion molecule and a pharmaceutically acceptable carrier. In another embodiment, there is provided a pharmaceutical composition comprising a bifunctional fusion molecule, an antigenic agent and a pharmaceutically acceptable carrier. In a further embodiment, there is provided a bifunctional fusion molecule, an antigenic agent and a kit. The pharmaceutical composition, as described herein, may be used to immunize a subject.

Kits and commercial packages containing the bifunctional fusion molecules and/or the antigenic agents described herein or kits and commercial packages containing a pharmaceutical composition as described herein, are contemplated. Such a kit or commercial package will also contain instructions regarding use of the included bifunctional fusion molecules, antigenic agents and/or pharmaceutical compositions, for example, to treat infectious diseases in accordance with the methods described herein.

In one aspect, a plurality of bifunctional fusion molecules directed to distinct cell surface antigens may be administered to a subject in combination with a plurality of distinct antigenic agents, wherein the antigenic agents associate with the moiety of the second functional domain of the bifunctional fusion molecule.

In another aspect, a plurality of bifunctional fusion molecules directed to distinct cell surface antigens may be administered to a subject in combination with a plurality of identical antigenic agents, wherein the antigenic agents associate with the moiety of the second functional domain of the bifunctional fusion molecule.

In another aspect, a plurality of bifunctional fusion molecules directed to the same cell surface antigen may be administered to a subject in combination with a plurality of distinct antigenic agents, wherein the antigenic agent associates with the moiety of the second functional domain of the bifunctional fusion molecule.

In a further aspect, a plurality of bifunctional fusion molecules directed to the same cell surface antigen may be administered to a subject in combination with a plurality of identical antigenic agents, wherein the antigenic agent associates with the moiety of the second functional domain of the bifunctional fusion molecule.

As used herein, “immunize” or “immunization” and “vaccinate” or “vaccination” are used interchangeably and refer to a means for providing protection against a pathogen by inoculating a host with an immunogenic preparation containing a bifunctional fusion molecule and an antigenic agent, in combination with an APC co-stimulatory molecule, such as a CD40 agonist, such that the host immune system is stimulated and prevents or attenuates subsequent pathology associated with the host reactions to subsequent exposures of the pathogen. Alternatively, a host may be inoculated solely with an immunogenic preparation containing a bifunctional fusion molecule and an antigenic agent to induce tolerance in treating an autoimmune disease.

A person skilled in the art would know how to prepare suitable vaccine formulations. Conventional procedures and ingredients for the selection and preparation of suitable formulations are described, for example, in Remington's Pharmaceutical Sciences (16th edition, Osol, A. Ed. (1980) Mack Printing Company, Easton, Pa.) and in The United States Pharmacopeia: The National Formulary (USP 24 NF19) published in 1999.

To aid in administration of such an antibody and/or antigenic agent to a subject, such as a subject in need of treatment of an infectious disease, an antibody and/or antigenic agent may be formulated as an ingredient in a pharmaceutical composition.

The pharmaceutical composition may further include a pharmaceutically acceptable diluent or carrier. The invention in one aspect therefore also includes such pharmaceutical compositions for use in treating an infectious disease. The compositions may routinely contain pharmaceutically acceptable concentrations of salt, buffering agents, preservatives and various compatible carriers. Pharmaceutically acceptable carriers, diluents and excipients are known in the art and are described, for example, in Remington's Pharmaceutical Sciences 16th edition, Osol, A. Ed. (1980) Mack Printing Company, Easton, Pa.

The forms of the pharmaceutical compositions suitable for injectable use include sterile aqueous solutions or dispersion and sterile powders for the extemporaneous preparation of sterile injectable solutions or dispersions, wherein the term sterile does not extend to any cell that may comprise the pharmaceutical product of interest that is to be administered. In all cases the form must be sterile and must be fluid to the extent that easy syringability exists.

The dose of the pharmaceutical composition that is to be used depends on the particular condition being treated, the severity of the condition, individual patient parameters including age, physical condition, size and weight, the duration of the treatment, the nature of concurrent therapy (if any), the specific route of administration and other similar factors that are within the knowledge and expertise of the health practitioner. These factors are known to those of skill in the art and can be addressed with minimal routine experimentation. Typical dosages of the bifunctional fusion molecule and/or the antigenic agent is about 1 mg, about 750 μg, about 500 μg, about 250 μg, about 100 μg, about 10 μg, about 1 μg, about 750 ng, about 500 ng, about 250 μg, about 100 ng, about 50 ng, about 10 ng, about 1 μg, about 750 pg, about 500 pg, about 250 pg or about 100 pg.

The pharmaceutical composition may be administered to a subject in a variety of forms depending on the selected route of administration, as will be understood by those skilled in the art. The composition of the invention may be administered orally, by injection (intramuscular, intradermal, subcutaneous, intraperitoneal), by puncture, transdermally or intranasally.

The bifunctional fusion molecule may be conjugated to a moiety to reduce its immunogenicity or to increase its circulating half-life. In one embodiment, the bifunctional fusion molecule is conjugated to polyethylene glycol (PEG). PEG refers to an oligomer or polymer of ethylene oxide. PEGs are prepared by polymerization of ethylene oxide and are commercially available over a wide range of molecular weights from 300 g/mol to 10,000,000 g/mol. Polyethylene glycol has a low toxicity. PEG attached drugs, such as IFN-α show reduced immunogenicity and decreased clearance resulting in longer circulating half-life in vivo.

In another embodiment, the immunogenicity and/or half-life of the bifunctional fusion molecule and/or the antigenic agent may be modified using a nanoparticle or liposome.

In a further embodiment, the immunogenicity and/or half-life of the bifunctional fusion molecule and/or antigenic agent may be modified by lipidization of the bifunctional fusion molecule and/or the antigenic agent (Yuan et al., J. Controlled Release, 129: 11-17, 2008).

The pharmaceutical compositions of the present invention may be useful in the therapeutic and/or prophylactic treatment of infectious diseases and/or in modulating the immune response to an antigen. In one embodiment the pharmaceutical composition, in the absence of a co-stimulatory molecule, may be used for the treatment of an autoimmune disease. In another embodiment, the pharmaceutical composition, in the absence of a co-stimulatory molecule, may be used for the treatment of organ transplant.

As used herein, “treat” or “treatment” refers to an approach for obtaining beneficial or desired results, including clinical results. Beneficial or desired clinical results can include, but are not limited to, alleviation or amelioration of one or more symptoms or conditions, diminishment of extent of disease, stabilization of the state of disease, prevention of development of disease, prevention of spread of disease, delay or slowing of disease progression, delay or slowing of disease onset, amelioration or palliation of the disease state, and remission (whether partial or total). “Treating” can also mean prolonging survival of a subject beyond that expected in the absence of treatment. “Treating” can also mean inhibiting the progression of disease, slowing the progression of disease temporarily, although more preferably, it involves halting the progression of the disease permanently.

The pharmaceutical composition of the present invention may be useful for modulating the immune response of a subject to an antigen. As used herein, “modulate” or “modulating” may refer to increasing the strength, magnitude and/or duration of the immune response, or to suppressing the immune response resulting in unresponsiveness to the antigen.

In one embodiment, the subject is administered a bifunctional fusion molecule, an antigenic agent and a CD40 agonist such as an anti-CD40 antibody or another co-stimulatory molecule, inducing the immune response of the subject to the antigen. In another embodiment, the subject is administered a bifunctional fusion molecule and an antigenic agent but without a CD40 agonist such as an anti-CD40 antibody or a co-stimulatory molecule, thus suppressing, anergizing or inactivating the immune response of the subject to the antigen.

The invention is further illustrated by the following, non-limiting examples.

### EXAMPLES

### Materials and Methods

### Materials

DC 2.4 is a DEC-205 expressing mouse bone marrow dendritic cell-line transduced with GM-CSF, myc and raf oncogenes [Shen et al. J Immunol, 158:2723-30, 1997]. HB290, a rat anti-mouse DEC-205 hybridoma, was obtained from ATCC. BSA (bovine serum albumin), streptavidin-HRPO (horseradish peroxidase), anti-His₆ mAb (monoclonal antibody), NHS-LC-Biotin (biotinamidohexanoic acid 3-sulfo-N-hydroxysuccinimide ester), photobiotin acetate, OVA and goat-anti-mouse-HRPO (GAM-HRPO) were from Sigma (Oakville, Canada). Biotinylated MUC-1 peptide with amino sequence of B-GVTSAPDTRGVTSAPDTR(N-terminal biotinylated) was kindly provided by Biomira, Inc. (Edmonton, Alberta, Canada). The streptavidin gene was kindly provided by Dr. T. Sano, Center for Molecular Imaging, Diagnosis and Therapy and Basic Science Laboratory, Boston, Mass., USA. HSF (hybridoma serum free media), DMEM, PSG (penicillin, streptomycin and L-glutamine) and FBS (fetal bovine serum) were purchased from Gibco BRL (Burlington, Canada). B-BSA [(biotin), labeled BSA] was prepared by biotinylation of BSA with NHS-LC-Biotin as per vendor's protocol. TMB (3, 3′, 5, 5′-tetramethylbenzidine) peroxidase substrate was purchased from Kirkegaard & Perry Laboratory Inc (Gaithersburg, USA). Hybond ECL (enhanced chemiluminiscent) nitrocellulose membrane and the ECL Western blotting kit were from Amersham Pharmacia Biotech (Baie dUrfe, Canada). The E. coli strain BL21-CodonPlus® (DE3)-RIPL and pBlueScript II KS-(PKS) were purchased from Stratagene (Cedar Creek, USA). T7 promoter and terminator primers and the expression vector pET-22b (+) were from Novagen (Madison, USA). pVAX1 mammalian expression vector and molecular cloning materials (TOP10 cells, FastTrack mRNA isolation kit, modifying and restriction enzymes) were from Invitrogen (Burlington, Canada). Protein assay reagent was purchased from Bio-Rad (Mississauga, Canada). Ni-NTA agarose was purchased from Qiagen (Mississauga, Canada). Mouse IFN-γ (Interferon gamma) ELISA Ready-SET-Go was purchased from eBioscience (San Diego, USA). LAL (limulus amebocyte lysate) PYROGENT® Plus Single Test Vials was purchased from Cambrex (Walkersville, USA).

Rat anti-mouse CD40 mAb was prepared from the hybridoma IC10, kindly provided by Dr. M. Gold (University of British Columbia, Canada). pVHX-6, WEEV DNA encoding E1 and E2 proteins was provided by Dr. L. Nagata (Chemical Biological Defense Section, Defence R&D Canada) [Nagata et al. Vaccine, 23:2280-3, 2005]. SARS-CoV membrane codon optimized DNA was purchased from GENEART. EBOV GP1,2 DNA [Wahl-Jensen et al. J Virol, 79:10442-50, 2005], EBOV GP1,2 mammalian expressed protein and SARS-CoV spike DNA were from Dr. S. Jones and Dr. T. Booth (Special Pathogens Program, National Microbiology Laboratory, Canada). Gangliosides and ganglioside conjugates were from Dr. D. Bundle (University of Alberta, Canada). Gangliosides GM2 and GM3 antigens were biotinylated (B-GM2, B-GM3) and conjugated with BSA (B-BSA-GM2, B-BSA-GM3). Anthrax Protective Antigen (PA), extracted from the S-layer of recombinant Caulobacter crescentus, was provided by Dr. J. Smit (University of British Columbia, Canada).

### Cells and Antigen Preparation

DC 2.4 was cultured at 37° C. with 5% CO₂ in DMEM-10 medium [10% (v/v) FBS and 1% (v/v) PSG]. HB290 hybridoma was cultured under the same conditions in HSF medium [1% (v/v) FBS and 1% (v/v) PSG]. The various primers and antigens used for all the experiments are listed in Tables 1A and 1B. SARS-CoV spike DNA (encoding S1, S2, RBD and transmembrane domain), SARS-CoV membrane DNA, EBOV GP1,2 DNA were PCR (polymerase chain reaction) amplified using primers WP011 [SEQ ID NO: 11] and WP012 [SEQ ID NO: 12], WP013 [SEQ ID NO: 13] and WP014 [SEQ ID NO: 14], or WP015 [SEQ ID NO: 15] and WP016 [SEQ ID NO: 16] respectively (Table 1A). These primers inserted Kozak translation initiation sequence and initiation codon (ATG) into the restriction sites BamHI, EcoRI. The PCR fragments were gel-purified, double digested with BamHI and EcoRI, ligated to pVAX1 mammalian expression vector and transformed into TOP10 cells. The positive clones were screened by restriction digestion fragment mapping (BamHI and EcoRI) and then biotinylated using photobiotin acetate [McInnes et al. Methods Enzymol. 184:588-600, 1990]. Core-streptavidin, WEEV E1, WEEV E2 and EBOV GP1 (Subfragment D) were prepared based on previous work [Das et al. Protein Expr Purif, 54:117-25, 2007; Wang et al. Mol Biotechnol, 31:29-40, 2005; Das et al. Virus Res, 128:26-33, 2007; Das et al. Antiviral Res, 64:85-92, 2004]. EBOV GP2, SARS-CoV spike S1, S2, RBD and membrane proteins were produced from E. coli expression system. All antigens for immunization were labelled with biotin. DNA vectors were biotin labelled using photobiotin acetate (B-pVHX-6, B-pEBOV GP1,2, B-pSARS-CoV spike, B-pSARS-CoV membrane), all proteins were labelled with NHS-LC-Biotin (B-OVA, B-SARS-CoV spike RBD, B-EBOV GP1, B-Anthrax PA), glycolipids (B-GM2, B-GM3, B-BSA-GM2, B-BSA-GM3) and peptide (B-MUC-1) were synthetically labelled with biotin. Biotinylation of all antigens was confirmed by dot blot assay probed with streptavidin-HRPO.

### Sequencing and Cloning of HB290 Gene

### Sequencing of HB290 Fab

1×10⁹ HB290 cells were harvested and the mRNA was isolated using FastTrack® mRNA isolation kit according to vendor's protocol. PCR primers were designed based on protocols in Methods in Molecular Biology volume 178 [O'Brien and Aitken. Methods in molecular biology, vol. 178: antibody phage display: methods and protocols, Humana Press, 2002]. cDNA of mAb from the variable regions (V_{L} and V_{H}) to the constant light chain region (C_{L}) and heavy chain region (CH₂) were generated by RT-PCR (reverse transcription polymerase chain reaction) and TD-PCR (touch down polymerase chain reaction) using rat specific PCR primers and inserted in PKS plasmid. Positive clones were selected by restriction digestion mapping and DNA sequencing. The positive cloned fragment was sequenced using M13 primers by CEQ™2000 (Beckman Coulter, USA). DNA alignment between the forward sequence and backward sequence was done using DS Gene 1.1 software (Discovery Studio). The sequence generated after DNA alignment was compared with the known mAb sequences in NCBI BLAST databank. The consensus sequence of 2 individual clones was selected from two independent PCR resulted in 100% match in both nucleotide sequence and amino acid sequence.

Cloning of HB290 scFv and bfFp

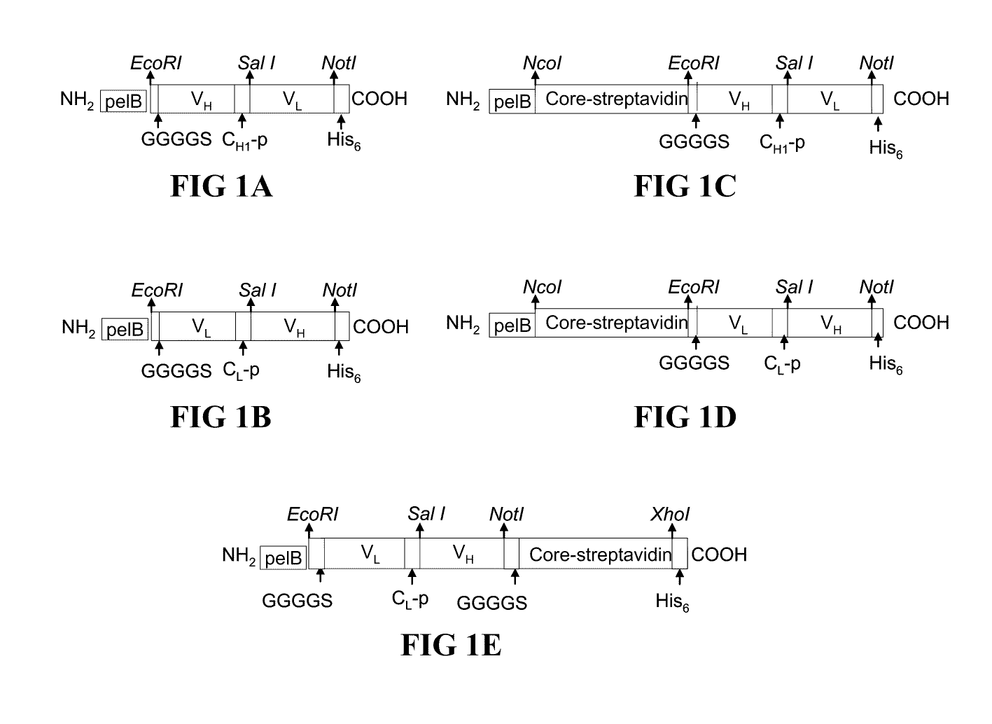

The various cloning constructs of HB290 scFv and bfFp are listed in FIG. 1. The genes were cloned in different orientations to determine the best expression in E. coli. The heavy and light chain variable regions with/without partial constant regions of anti-DEC205 antibody were amplified from PKS plasmid containing HB290 Fab DNA by PCR and cloned into pET-22b (+) plasmid. The heavy chain variable region gene of HB290 was fused to the 3′ end of light chain variable region gene with a linker of 15 amino acids from constant heavy chain region 1 sequence using PCR (PCR primers: WP005-WP008). Subsequently restriction digest/ligation methods were employed to generate V_{H}-V_{L} scFv gene (FIG. 1A). The PCR primers WP005 [SEQ ID NO: 5] and WP006 [SEQ ID NO: 6] were inserted into the restriction sites EcoRI and SalI; PCR primers WP007 [SEQ ID NO: 7] and WP008 [SEQ ID NO: 8] were inserted into the SalI and NotI. The PCR fragments were gel-purified, double digested to the respective inserted restriction sites, and ligated to pET-22b (+) plasmid. V_{L}-V_{H} scFv gene was generated also by the same methods utilizing PCR primers WP001-WP004 (WP001 [SEQ ID NO: 1] and WP002 [SEQ ID NO: 2] were inserted into the EcoRI and SalI; WP003 [SEQ ID NO: 3] and WP004 [SEQ ID NO: 4] were inserted into the SalI and NotI). The light chain variable region gene of HB290 was fused to the 3′ end of heavy chain variable region gene via 15 amino acids of constant light chain sequence (FIG. 1B). Both scFv orientations were inserted into pET-22b (+) containing the core-streptavidin gene [Wang et al. Eur J Pharm Biopharm, 65:398-405, 2007] in 3′ orientation fusion with the core-streptavidin gene (FIGS. 1C and 1D). V_{L}-V_{H} scFv gene was fused in 5′ terminus with core-streptavidin gene by the same method described above (FIG. 1E). Briefly, core-streptavidin gene was PCR amplified using WP009 [SEQ ID NO: 9] and WP010 [SEQ ID NO: 10] primers. These primers were inserted into restriction sites NotI and XhoI. The PCR fragment was gel-purified, double digested with NotI and XhoI, and ligated to pET-22b (+) containing V_{L}-V_{H} scFv gene (FIG. 1E). All clones were screened and characterized by both PCR and restriction digestion fragment mapping. The positive cloned fragment was sequenced using T7 promoter and terminator primers by CEQ™2000. The positive clones were named as follows: WET5 encoding core-streptavidin-V_{H}-V_{L}, WET6 encoding core-streptavidin-V_{L}-V_{H} and WET7 encoding V_{L}-V_{H}-core-streptavidin.

### Expression, Purification and Characterization of HB290 Recombinant Proteins

Expression of HB290 scFv and bfFp

Expression of scFv and bfFp methods and conditions were previously described [Wang et al. Eur J Pharm Biopharm, 65:398-405, 2007]. Briefly, the pET-22b (+)-scFv or bfFp genes (WET5-7) were chemically transformed into BL21-CodonPlus® (DE3)-RIPL. E. coli transformants were cultured and induced and the whole-cell bacterial pellets were analyzed by Western blot. The pellets were resuspended in reducing SDS dye (50 mM Tris-HCl, pH 6.8, 2% SDS, 0.1% bromophenol blue, 10% glycerol, 5 mM 2-mercaptoethanol) and heated at 95° C. for 10 min prior to SDS-PAGE. The pellets were electrophoresed and transferred to a Hybond ECL nitrocellulose membrane using the Trans blot apparatus (Bio-Rad). The membrane was then blocked with 5% skim milk, probed with mouse anti-His₆ mAb and GAM-HRPO, and revealed by ECL according to the manufacturer's protocol. The bfFp gene with the highest protein expression level was selected for medium scale expression in culture flask. Affinity purification from E. coli periplasm using IMAC purification protocol was performed as described previously [Wang et al. Eur J Pharm Biopharm, 65:398-405, 2007]. The induced periplasm and IMAC purified fractions were analyzed by SDS-PAGE using 10% polyacrylamide gels under reducing conditions following staining with Coomassie brilliant blue. The fractions were heated at 95° C. for 10 min prior to loading on the polyacrylamide gel.

Western Blot: bfFp Biotin Binding and Heat Stability

IMAC purified bfFp was heated at either 60° C. or 95° C. for 10 min under reducing conditions and resolved in 10% SDS-PAGE. The resolved proteins were electrophoretically transferred onto a nitrocellulose membrane and probed with B-BSA followed by streptavidin-HRPO.

ELISA: bfFp Bispecificity and Binding to DC 2.4 Cells

DC 2.4 cells were seeded on a 96-well V-bottomed plate (Nunc, Denmark) in quadruplicate (1.0×10⁵ cells/well). The plates were washed with PBS (phosphate buffer saline) and blocked with 1% PBS dialyzed BSA (to remove traces of biotin) for 3 h at 4° C. After incubation, the plates were washed with PBS, and bfFp (40 μg/ml in 100 μl volume) was added to bind DEC-205 receptors. The plates were incubated at 4° C. for 3 h, then washed with PBS and B-OVA (20 μg/ml in 100 μl volume) was added to each well and incubated for 1 h at 4° C. After the incubation, the plates were washed and then incubated with streptavidin-HRPO (10 μg/ml in 100 μl volume) for 1 h at 4° C. Finally, the plates were washed with PBS, and TMB substrate was then added. The OD_{650 nm} was taken after 15 min using an ELISA Vₘₐₓ kinetic microplate reader (Molecular Devices Corp, California, USA) and subtracted with control (dendritic cells with only streptavidin-HRPO incubated at 4° C. for 1 h). The bfFp DEC-205 receptor binding specificity was confirmed by competition study with full-length HB290 mAb. Various concentrations of HB290 mAb (50, 100, 200, 300 μg/ml in 100 μl volume) was added after bfFp binding to DEC-205. The mAb was incubated for 2 h at 4° C. then washed with PBS and the bfFp was detected using B-OVA, streptavidin-HRPO and TMB as mentioned above.

### In Vivo Targeting of Dendritic Cells in Mice

### Mice and Immunization Protocol

15 groups of female BALB/c mice (5 mice per group, average 6-8 weeks old) were obtained from Health Sciences Laboratory Animals Services of the University of Alberta, Edmonton, Canada. Animal treatment, care and euthanasia were carried out according to the Canadian Council of Animal Care guidelines. Mice were injected subcutaneously near the inguinal lymph node area with 0.1 ml of various formulations of antigens and bfFp in saline. The immunization protocol is listed in Table 2. Three separate experiments were conducted and each design has its own control group. All groups had n=5 mice. The first set of experiments demonstrated the versatility of bfFp based delivery of protein, peptide, glycolipids and DNA to dendritic cells in generating immune responses. B-pVHX-6, B-OVA, B-MUC-1, B-GM2 & GM3 were studied (Groups 2-7 mice). Group 2-4 mice focused on the DNA delivery to dendritic cells and to verify the essential requirement of dendritic cell targeting vehicle and co-stimulatory molecule. Group 2 mice were immunized with B-Pvhx-6 and bfFp without the presence of anti-CD40 mAb to verify the essential role of anti-CD40 mAb co-stimulation on dendritic cells. Group 3 mice were co-immunized with anti-CD40 mAb without the bfFp to determine the non-targeted immune responses. Group 4 mice were co-immunized with both bfFp and anti-CD40 mAb. Protein antigen delivery is studied in Group 5 mice, peptide delivery is focused in Group 6 mice, and glycolipids were investigated in Group 7 mice. The second group of experiments was designed to confirm the DNA delivery strategy. A variety of infectious disease viral DNA were targeted to dendritic cells using bfFp mediated delivery system (Group 9, EBOV GP1,2 DNA; Group 10, SARS-CoV spike DNA; Group 11, SARS-CoV membrane DNA). Multivalent immune responses against a mixture of antigens were studied in the third set of experiments. Groups 13-15 were designed to evaluate multivalent immune responses against proteins and peptide. B-OVA, B-EBOV GP1, B-SARS-CoV spike RBD, B-MUC-1, B-Anthrax PA were immunized as a mixture in saline in Groups 13-15 mice. Group 13 mice were co-immunized with anti-CD40 mAb and core-streptavidin without the bfFp to determine the non-targeted immune responses. Group 14 mice were co-immunized with bfFp without the presence of anti-CD40 mAb to verify the essential role of anti-CD40 mAb co-stimulation. Group 15 mice were immunized with antigens, bfFp and anti-CD40 mAb. Groups 1, 8 and 12 are the control groups for the three separate animal experiments. All mice were boosted with the same concentration of antigen(s) in PBS 12 days following primary immunization. Prior to immunization, every reagent (antigens, bfFp and mAb) was checked using LAL PYROGENT® Plus Single Test Vials kits to identify lipopolysaccharide (LPS) contamination (endotoxin sensitivity at 0.125 EU/ml). The mice were sacrificed after 9 days of boost. The blood and the spleen were collected. The serum was isolated using standard procedure [Coligan et al. (Eds.) Current Protocols in Immunology 1.7.1-1.7.8. Wiley, 1995] and used to evaluate humoral immune responses. Spleens were used to study IFN-γ immune responses. The spleens from the 15 different groups of immunized mice were aseptically removed and each group was pooled. The responder cells were isolated using nylon wool columns and the stimulator cells from the naïve mouse spleens were treated with mitomycin C as previously described [Wang et al. J Immunol Methods, 306:80-92, 2005].

### Evaluation of Humoral Immune Responses and IFN-γ

Antibody titres were measured individually in each mouse by ELISA following immunization of the respective antigen (Table 1B). The ELISA method was done by overnight coating of the specific antigen in the Nunc 96-well ELISA microplates (10 μg/ml in 100 μl volume). After overnight coating, the plates were washed with PBST (0.1% Tween 20 in PBS, pH 7.3) and the plates were blocked with BSA for 3 h at RT. After incubation, the plates were washed with PBST and the 1:1000 serially diluted serum from each mouse in quadruplicate was added and incubated for 2 h at RT. Plates were then washed with PBST and incubated with GAM-HRPO for 1 h at RT. After 1 h, the plates were washed again with PBST and TMB was then added to each well and OD_{650 nm} was taken after 10 min using a microplate reader. Statistical analyses were performed as described [Wang et al. J Immunol Methods, 306:80-92, 2005] to determine the significance between antibody responses in different groups. IFN-γ activity was determined by the IFN-γ concentration generated after 3 days of incubation of responder cells (2.5×10⁵ cells) and/or stimulator cells (3×10⁵ cells) with the respective antigens: OVA, SARS-CoV spike RBD, EBOV GP1, EBOV GP2, EBOV GP1,2, SARS-CoV spike 51, SARS-CoV spike S2, SARS-CoV spike RBD, SARS-CoV membrane, Anthrax PA, B-MUC-1, B-GM2, B-GM3, WEEV E1 and WEEV E2 (10 μg per antigen, each antigen is separately incubated). IFN-γ ELISA Ready-SET-Go kit was used to determine the IFN-γ concentration.

Immune Responses Towards Biotin, bfFp and Core-Streptavidin

All the 15 groups of mice serum were tested against B-BSA, bfFp and core-streptavidin using ELISA method. The serum reactivity was determined by the same method as described above. The plates were coated with B-BSA, bfFp or core-streptavidin (10 μg/well). The rest of procedures and statistical analyses were as enumerated in the above section.

### Results

Construction, Expression and Purification of HB290 scFv and bfFp

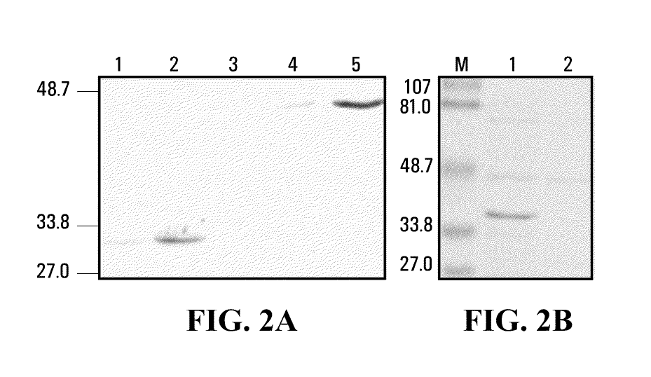

The genes encoding HB290 Fab were generated by RT-PCR and TD-PCR. The sequence generated was compared with the known mAb sequences in NCBI BLAST database indicating the sequence is antibody related. HB290 V_{L} amino acid sequence from the recombinant clone was 100% identical to a previous publication on cloning of NLDC-145 (HB290) scFv [Demangel et al. Mol Immunol, 42:979-85, 2005]. However, HB290 V_{H} deduced amino acid sequence (EVKLVESGGGLVQPGGSLRLSCAASGFTFNDFYMNWIRQPPGQAPEWLGVIRNKGNG YTTEVNTSVKGRFTISRDNTQNILYLQMNSLRAEDTAIYYCARGGPYYYSGDDAPYWGQ GVMVTVSS-SEQ ID NO: 17) shared only 46% homology. The V_{H} amino acid sequence appears to be unique from other published amino acid sequences in the NCBI protein database; whereas, the amino acid sequence published by Demangel and co-workers is 99% identical (only 2 amino acid difference in the 5′ terminal) with single chain antibody against rice stripe virus protein P20 (Accession: AAG28706). HB290 V_{L}-V_{H} and V_{H}-V_{L} genes were successfully cloned into separate pET-22b (+) plasmid by PCR, restriction digest and DNA ligation methods. The plasmid vectors WET5 and WET6 were successfully constructed by inserting the sequence encoding the HB290 single-chain anti-DEC205 antibody (V_{L}-V_{H} or V_{H}-V_{L} respectively) into a core-streptavidin containing pET22b (+) plasmid next to the pelB leader sequence (FIGS. 1A-D). The plasmid vector WET7 was constructed by inserting the core-streptavidin sequence into HB290 V_{L}-V_{H} scFv containing pET22b (+) plasmid (FIGS. 1B and 1E). The core-streptavidin sequence is at the N-terminus of the scFv in WET5 and WET6 constructs; whereas, in WET7 the core-streptavidin is in the C-terminal. The scFv and bfFp vectors were transformed into E. coli, cultured, induced and the whole-cell bacterial pellets were analyzed by Western blot using the anti-His₆ mAb (FIG. 2A). The optimal scFv and bfFp orientation for expression were determined by the Western blot. HB290 V_{L}-V_{H} scFv, WET6 and WET7 were successfully expressed and the proteins are shown at the desired MW (molecular weight) band either at ˜30 kDa or ˜46 kDa (FIG. 2A). WET7 had higher level of protein expression compare to WET6. WET7 was subjected to medium scale expression, and the bfFp in the periplasmic space was extracted and affinity purified by IMAC column. Both periplasmic fraction and the affinity purified fraction were analyzed on SDS-PAGE (FIG. 2B). IMAC purification of the WET7 bfFp was successful; a clear band is shown at 46 kDa in affinity purified fraction (FIG. 2B). Approximately 1.0 mg of WET7 bfFp was affinity purified from a 2 L culture.

Characterization of bfFp

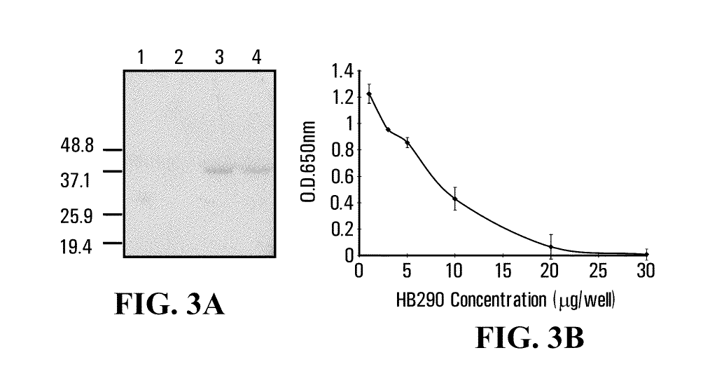

E. coli expressed bfFp appears in 3 isoforms: monomeric, dimeric and tetrameric forms similar to a published report [Kipriyanov et al. Protein Eng, 9:203-11, 1996]. Following affinity purification by either IMAC or iminobiotin column the fusion protein was subjected to size exclusion chromatography for isolation of the tetrameric fusion protein [Kipriyanov et al. Protein Eng, 9:203-11, 1996; Schultz et al. Cancer Res, 60:6663-9, 2000]. The fusion protein is known to be heat sensitive and heating at 95° C. dissociates the tetramer completely into monomeric forms. The tetrameric form remains stable at temperature below 60° C. [Kipriyanov et al. Protein Eng, 9:203-11, 1996]. The bfFp isoforms and the biotin binding activity of the bfFp were analyzed by Western blot probed with B-BSA (FIG. 3A). WET7 bfFp was detected using B-BSA and streptavidin-HRPO in Western blot. The bfFp appears predominantly in monomeric form after heating at either 60° C. or 95° C. (FIG. 3A). The predominant monomeric form may be due to the difference in the linker between core-streptavidin and scFv [Wang et al. Eur J Pharm Biopharm, 65:398-405, 2007].

The bispecificity of the bfFp was confirmed by cell ELISA. The anti-DEC-205 activity was demonstrated on DC 2.4 cells employing B-OVA with streptavidin-HRPO for detection. In addition, specific DEC-205 receptor binding activity was also confirmed by competitive displacement of bfFp with increasing concentrations of HB290 mAb (FIG. 3B).

bfFp Mediated Immune Responses: Humoral and IFN-γ

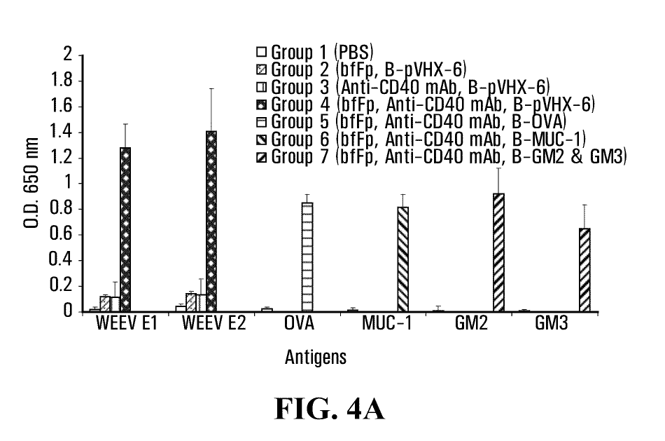

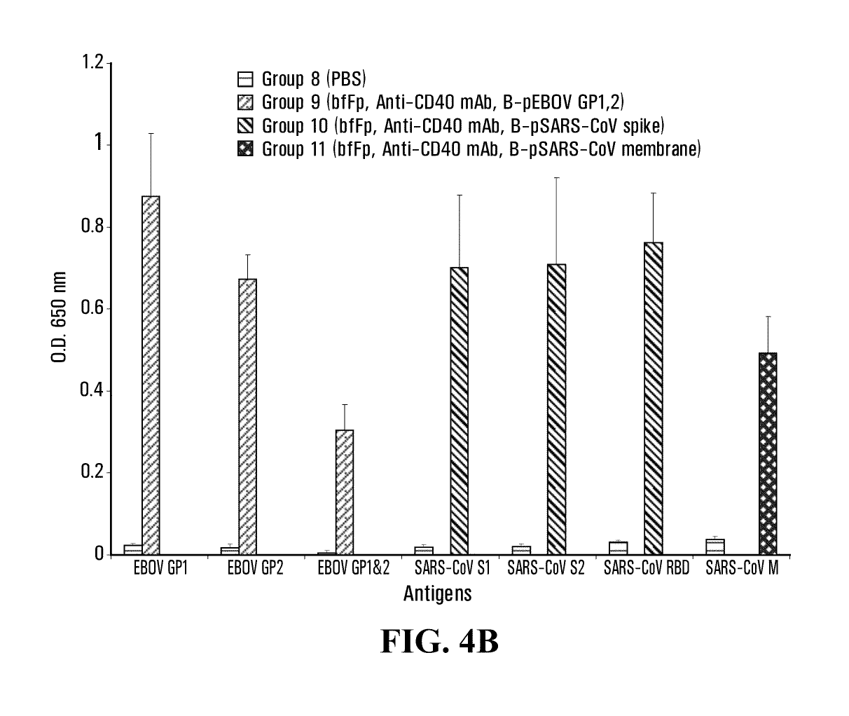

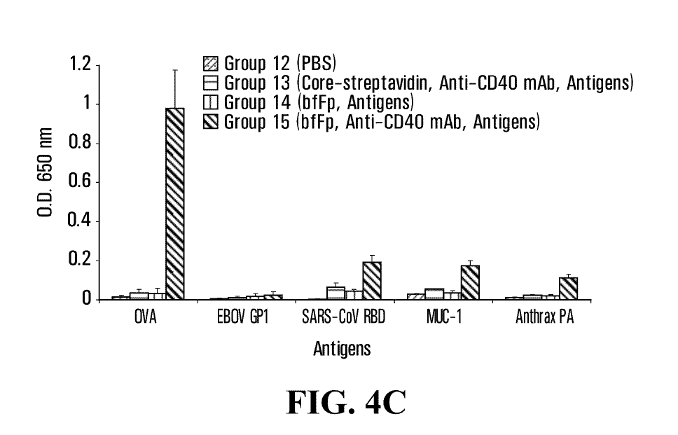

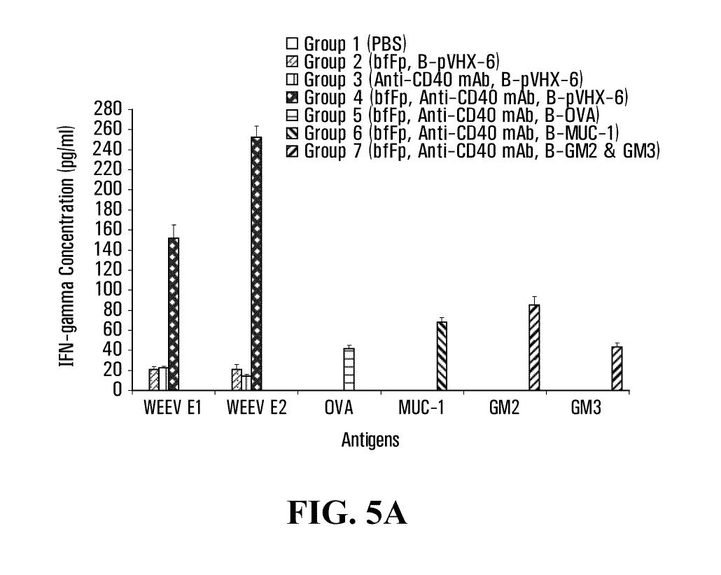

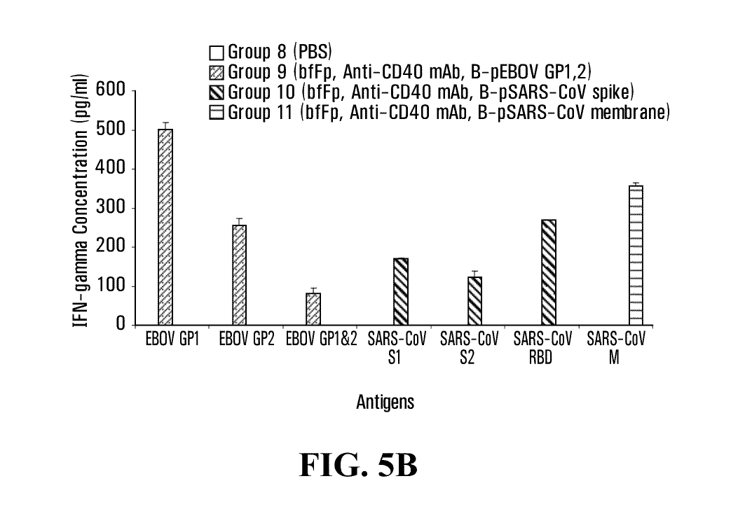

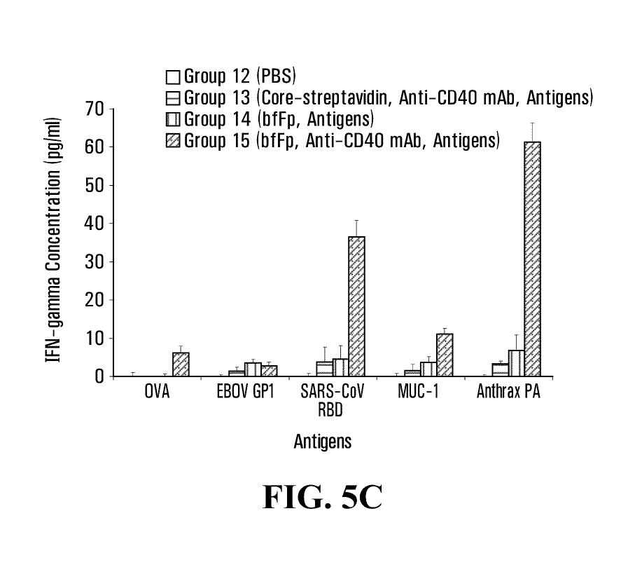

In vivo studies were carried out to investigate the ability and efficacy of bfFp targeting of antigens to dendritic cells. Four classes of antigens were chosen to demonstrate the versatility and diversity of antigen delivery. These antigens were either made by recombinant DNA technology or obtained from several sources. LPS is identified in E. coli expressed proteins (SARS-CoV proteins, EBOV proteins, WEEV proteins, bfFp), OVA and Anthrax PA. DNA vectors, MUC-1 peptide, anti-CD40 mAb and mammalian expressed GP1,2 were LPS free. Both humoral and cell-mediated responses were investigated using a variety of antigens listed in Table 1B. The immunization protocol is described in detail in Table 2 and the results of humoral and cell-mediated responses are shown in FIGS. 4 and 5. Humoral responses were measured by the antibody titres against the immunized antigens or its respective proteins from DNA vectors individually in each mouse by ELISA. The magnitude of cell-mediated immune response is determined by the amount of IFN-γ secreted from spleen T cells in response to the antigens. Individual mouse spleen was not studied since the humoral response data was reproducible from five individual mice. The spleen cells were pooled to average the quality of data. Responder cells from both immunized and non-immunized mice without stimulator cells had minimal IFN-γ secretion. 15 groups of mice were divided into three different experiments to demonstrate the diversity and efficacy of dendritic cell targeting strategies. Groups 1, 8 and 12 are the control groups for the three separate animal experiments, being only immunized and boosted with PBS. Both humoral and cell-mediated immune responses are minimal (FIGS. 4 and 5).

bfFp Mediated Protein, Peptide, Gangliosides and DNA Targeting

Groups 2-7 were designed to demonstrate the versatility of bfFp based delivery of protein, peptide, glycolipids and DNA to dendritic cells in generating immune responses. Group 2-4 mice focused on the DNA delivery to dendritic cells and to verify the essential requirement of bfFp and anti-CD40 mAb. Group 2 mice were immunized with B-pVHX-6 and bfFp without the presence of anti-CD40 mAb. Group 3 mice were co-immunized with anti-CD40 mAb without the bfFp. Group 4 mice were co-immunized with both bfFp and anti-CD40 mAb. The results indicate that Group 4 has highest antibody titre (statistically significant) and augmented IFN-γ secretion against WEEV E1 and WEEV E2 proteins compare to Groups 1-3 (FIGS. 4A and 5A). Immune responses against WEEV E2 antigen appear to be higher than WEEV E1 in Group 4 (FIGS. 4A and 5A). Immune responses in Groups 2 and 3 are minimal towards WEEV E1 and E2 protein antigens, probably due to LPS, anti-CD40 mAb or bfFp effect (FIGS. 4A and 5A). Anti-CD40 mAb and bfFp appeared to be essential for DNA targeting strategy. The strategy was applied to protein (OVA), peptide (MUC-1) and glycolipids (GM2 and GM3). Essentially similar humoral responses and cell-mediated immune responses are shown in mice immunized with protein (Group 5), peptide (Group 6) and glycolipids (Group 7) in the presence of bfFp and anti-CD40 mAb (FIGS. 4A and 5A). In summary, anti-CD40 mAb and bfFp are required to achieve immune responses in dendritic cell delivery of DNA.

The efficiency of targeting different classes of antigens such as protein, peptide, glycolipid and DNA to dendritic cells has been demonstrated. However, one DNA targeting model may not be sufficient to document the successful DNA targeting to dendritic cells. Thus, the same targeting strategy for a variety of infectious disease DNAs was tested. Groups 9-11 were immunized with different DNA vectors encoding genes for different viral proteins: (Group 9, EBOV GP1,2 DNA; Group 10, SARS-CoV spike DNA; Group 11, SARS-CoV membrane DNA). pEBOV GP1,2 encodes EBOV GP1 and GP2 protein, pSARS-CoV spike encodes SARS-CoV S1, S2, RBD and transmembrane domain, and pSARS-CoV membrane encodes SARS-CoV membrane protein. Strong humoral and cell-mediated responses were achieved against the encoded viral proteins by targeting viral DNA to dendritic cells (FIGS. 4B and 5B). EBOV GP1 generates highest immune responses compare to GP2 and GP1,2. The immune responses against mammalian expressed GP1,2 protein are lower than E. coli expressed fragments (GP1 and GP2) (FIGS. 4B and 5B). Immune response against EBOV GP1,2 is relatively lower than its fragment may be due to the glycosylation masking of the epitopes for serum or T cell reactivity [Dowling et al. J Virol, 81:1821-37, 2007]. There is no significant difference in serum titer activity between SARS-CoV spike proteins, but higher IFN-γ concentration is found in SARS-CoV RBD. Thus the naked DNA delivery strategy was successful in several infectious diseases models.

bfFp Mediated Multiple Antigen Targeting Strategy

Groups 13-15 were designed to evaluate multivalent immune responses by targeting multiple biotinylated proteins and peptide antigen at same time in nanogram concentrations. Group 13 reflects the possible involvement of core-streptavidin targeting of a mixture of biotinylated proteins and peptide to dendritic cells. Groups 14 and 15 demonstrated the efficacy of the bfFp in the absence and presence of the anti-CD40 mAb co-stimulator respectively. The results show that Group 15 has the highest antibody titre (statistically significant) and augmented IFN-γ secretion against OVA, SARS-CoV RBD, Anthrax PA and MUC-1 compare to Groups 12-14 (FIGS. 4C and 5C). Groups 13-15 have shown minimal immune responses against EBOV GP1 (FIGS. 4C and 5C). In Group 15, highest antibody titre was found against OVA and lowest against Anthrax PA (FIG. 4C); whereas, highest IFN-γ secretion was found against Anthrax PA (FIG. 5C). Minimal humoral or cell-mediated immune responses generated from Groups 12-14 may be due to the anti-CD40 mAb, LPS and/or core-streptavidin effects (FIGS. 4C and 5C). MUC-1 and OVA immune responses were compared between single antigen and multiple antigen targeting strategies. Higher IFN-γ secretion and serum titre against MUC-1 were achieved in single antigen targeting strategy of MUC-1 peptide antigen (Group 6) in comparison to multiple antigens targeting strategy (Group 15) (FIGS. 4A, 4C, 5A, 5C). The serum titre against OVA is not different between Groups 5 and 15 (FIGS. 4A and 4C). However, Group 5 has higher IFN-γ secretion compared to Group 15 (FIGS. 5A and 5C). To summarize, both bfFp and anti-CD40 mAb are required to achieve strong immune responses against protein and peptide delivered to dendritic cells. Single antigen targeting appears to achieve strong humoral and cell-mediated immune responses in comparison to multi-antigen targeting strategy. Immune responses are variably shifted in multi-antigen targeting method, possibly towards immunodominant antigens.

Immunogenicity of bfFp

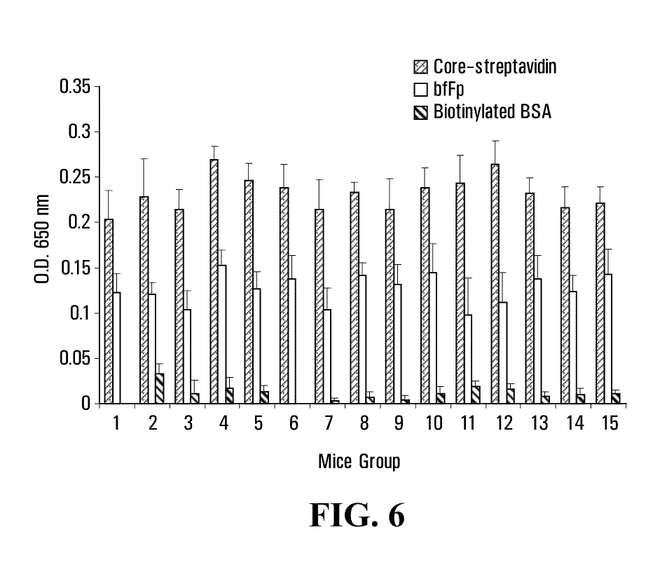

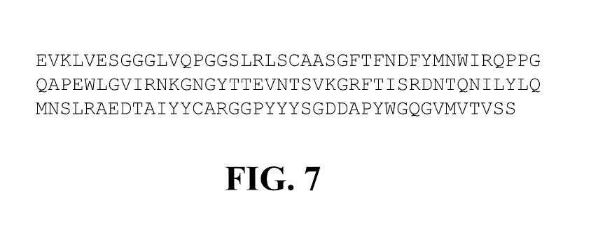

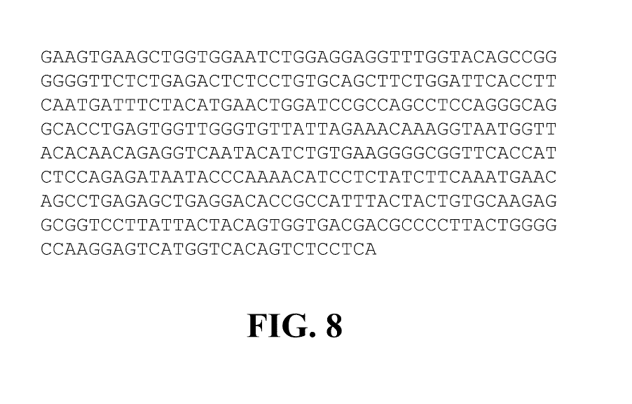

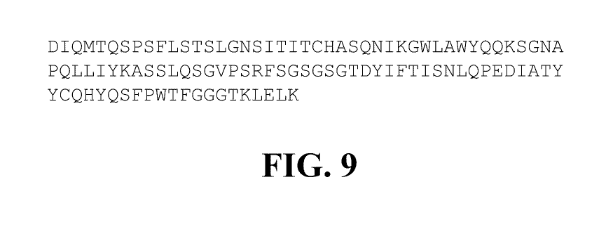

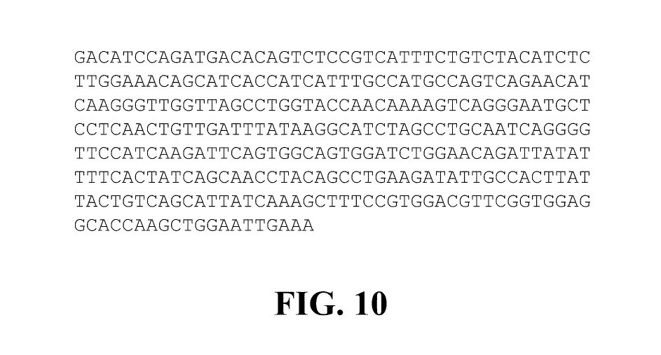

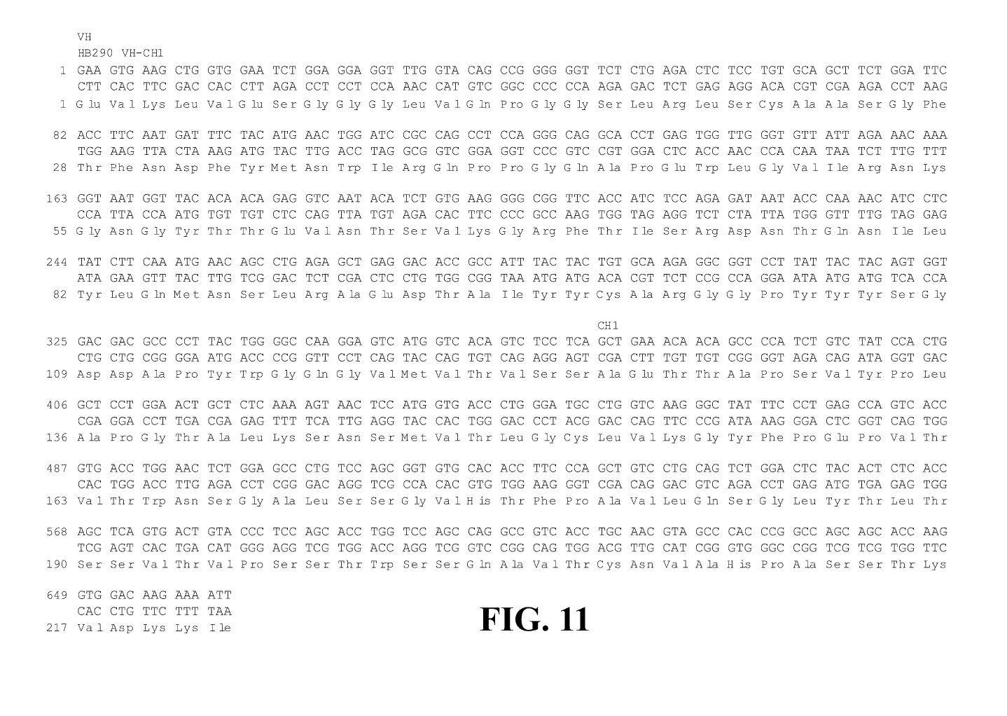

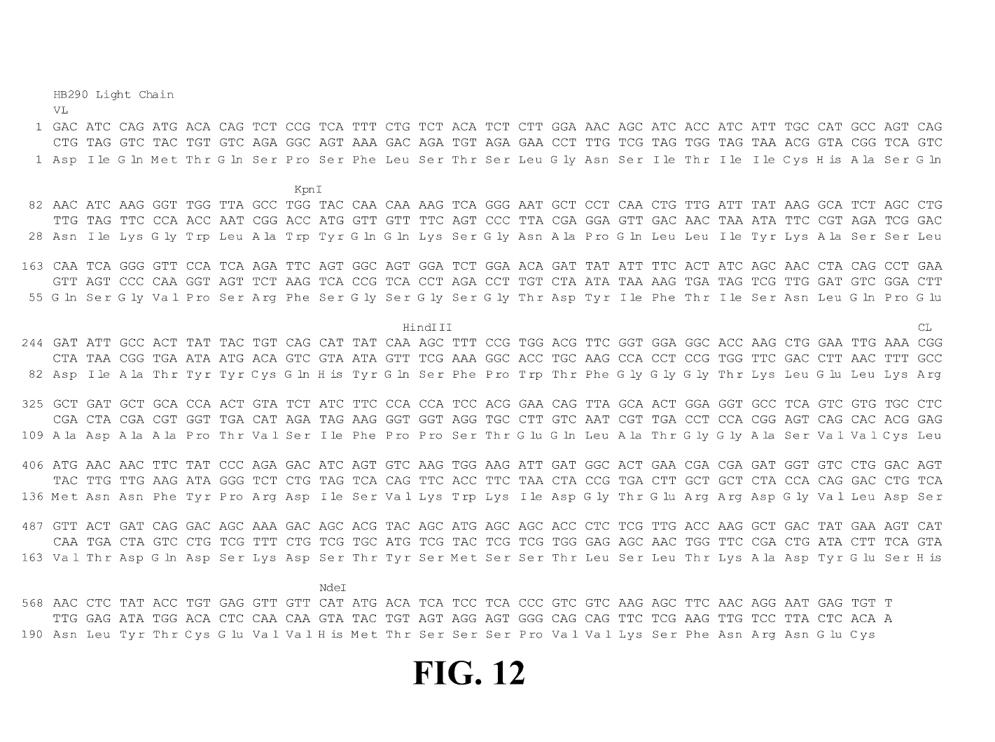

Serum titre against biotin, bfFp and core-streptavidin were analyzed from everyone of the 15 groups to evaluate the immunogenicity of the bfFp targeting vehicle. The bfFp, B-BSA and core-streptavidin proteins were coated on the ELISA plate. Serum reactivity was found to a minor extent against core-streptavidin (OD ˜0.25) and bfFp (OD ˜0.15). This level of serum reactivity is not significant compare to the control groups (FIG. 6). The serum reactivity towards B-BSA was minimal and not statistically significant between all groups (FIG. 6).

TABLE 1
A

| | | | |
|---|---|---|---|
| | | | SEQ |
| | | | ID |
| Primer | Function | Primer Sequence (5′-3′) | NO: |

| | | | |
|---|---|---|---|
| WP001 | 5′PCR primer, HB290 V_{L}-C_{L-p}(strep-V_{L}-V_{H}) | ATC AGT GAA TCC GGG AGG TGG CGG ATC AGA CAT CCA | 1 |
| | | GAT GAC ACA GTC T | |
| WP002 | 3′PCR primer, HB290 V_{L}-C_{L-p}(strep-V_{L}-V_{H}) | TGG TTT CGC TCA TGC TAG GTC GAC CGT GGA TGG TGG | 2 |
| | | GAA GAT AGA | |
| WP003 | 5′PCR primer. HB290 V_{H}(strep-V_{L}-V_{H}) | GTT AAT GTC GAC GAA GTG AAG CTG GTG GAA TCT | 3 |
| WP004 | 3′PCR primer, HB290 V_{H}(strep-V_{L}-V_{H}) | TAC TAA GCG GCC GCA AGC TGA GGA GAC TGT GAC | 4 |
| WP005 | 5′PCR primer, HB290 V_{H}-C_{H1-p}(strep-V_{H}-V_{L}) | ATC AGT GAA TTC GGG AGG TGG CGG ATC AGA AGT GAA | 5 |
| | | GCT GGT GGA ATC T | |
| WP006 | 3′PCR primer, HB290 V_{H}-C_{H1-p}(strep-V_{H}-V_{L}) | TGG TTT CGG TCA TGA TAG GTC GAC AGC AGT TCC AGG | 6 |
| | | AGC CAG T | |
| WP007 | 5′PCR primer, HB290 V_{L}(strep-V_{H}-V_{L}) | GTT AGG GTC GAC GAC ATC CAG ATG ACA CAG TCT | 7 |
| WP008 | 3′PCR primer, HB290 V_{L}(strep-V_{H}-V_{L}) | TAC TAA GCG GCC GCA AGC CCG TTT CAA TTC CAG C | 8 |
| WP009 | 5′PCR primer, core-streptavidin(V_{L}-V_{H}-strep) | TACTAATGCGGCCGCGGAGGTGGCGGATCAGAGGCCGGCATCACCGGCA | 9 |
| WP010 | 3′PCR primer, core-streptavidin(V_{L}-V_{H}-strep) | ATTACTCTCGAGGGAGGCGGCGGACGGCTTC | 10 |
| WP011 | 5′PCR primer, pSARS-CoV spike | AAGAGGGGGATCCTACCATGGGTAGTGACCTTGACCGGTGCACCACT | 11 |
| WP012 | 3′PCR primer, pSARS-CoV spike | CTCGCTCGAGAGAATTCTATTATGTGTAATGTAATTTGACACCCTTGAG | 12 |
| WP013 | 5′PCR primer, pSARS-CoV membrane | AAGAGGGTCTTCATATGGGGGATCCTACCATGGCAGACAACGGTACTAT | 13 |
| | | TACCGTTGAG | |
| WP014 | 3′PCR primer, pSARS-CoV membrane | CTCGCTCGAGAATTCTAGTGATGATGGTGGTGATGCTGTACTAGCAAAG | 14 |
| | | CAATATTGTCGTT | |
| WP015 | 5′PCR primer, pEBOV GP1, 2 | AAGAGGGGGATCCTACCATGGGCGTTACAGGAATATTGCAGTTACCT | 15 |
| WP016 | 3′PCR primer, pEBOV GP1, 2 | CTCGCTCGAGAGAATTCTAAAAGACAAATTTGCATATACAGAATAAAGC | 16 |

B

| Classification | Immunized antigens | Testing antigens |
|---|---|---|
| Protein | B-OVA | OVA |
| | B-SARS-CoV spike RBD | SARS-CoV spike RBD |
| | B-EBOV GP1 | EBOV GP1 |
| | B-Anthrax PA | Anthrax PA |
| Peptide | B-MUC-1 | B-MUC-1 |
| Glycolipid | B-GM2 | B-GM2 (IFN-γ Assay) |
| | B-GM3 | B-GM3 (IFN-γ Assay) |
| | | B-BSA-GM2 (Humoral study) |
| | | B-BSA-GM3 (Humoral study) |
| DNA | B-pVHX-6 | WEEV E1 |
| | B-pEBOV GP1, 2 | WEEV E2 |
| | B-pSARS-CoV spike | EBOV GP1 |
| | B-pSARS-CoV membrane | EBOV GP2 |
| | | EBOV GP1, 2 |
| | | SARS-CoV spike S1 |
| | | SARS-CoV spike S2 |
| | | SARS-CoV spike RBD |
| | | SARS-CoV membrane |

Table 1. (A) PCR primers for cloning of HB290 scFv (V_{L}-V_{H} and V_{H}-V_{L}) and bfFp (core-streptavidin-V_{H}-V_{L}, core-streptavidin-V_{L}-V_{H} and V_{L}-V_{H}-core-streptavidin orientations) into pET-22b (+) E. coli perplasmic expression vector. These primers contain the following restriction inserts: EcoRI and SalI for WP001, 002, 005 and 006; SalI and NotI for WP003, 004, 007 and 008; NotI and XhoI for WP009 and WP010. WP011-16 are the PCR primers for cloning of viral DNA into pVAX1 mammalian expression vector. These primers contain Kozak translation initiation sequence, initiation codon, and BamHI and EcoRI restriction sites. (B) Summary table for the antigen categories used for testing and targeted for in vivo study.

TABLE 1.1

| | | | Co-Stimulation | |
|---|---|---|---|---|
| Surface Receptors | Ligand | Distribution | Requirement | Applications |
| Gb3 (glycosphingolipid) | Shiga toxin | Monocytes, dendritic, endothelial, epithelial, and B | No | Vaccine vector |
| | | cells | | |
| β2 integrins (CD11c/CD18) | CyaA | Myeloid dendritic cells, macrophages, monocytes, | Yes | Vaccine vector |
| | | activated B cells, natural killer cells, granulocytes | | Tumor protection |
| CD40 receptors | CD40 ligand | Dendritic cells, B cells, macrophages, endothelial | No | Vaccine vector |
| | | cells, keratinocytes, fibroblasts, thymic epithelial | | Tumor protection |
| | | cells, CD34 hematopoietic cell progenitors | | Tumor therapy |
| | | | | Adenovirus delivery |
| C-type lectin receptors: | Unknown | Dendritic cells, Langerhans cells, monocytes, B | Yes | Vaccine vector |
| DEC-205 (CD205) | | cells, natural killer cells, T cells, respiratory tracts, | | Liposome delivery |
| | | thymic and gut epithelial cells | | Tumor protection |
| | | | | Tumor therapy |
| | | | | Infection protection |
| | | | | Suppress autoimmunity |
| C-type lectin receptors: | Hsp70 | Immature dendritic cells, macrophages, fibroblasts, | No | Vaccine vector |
| LOX-I (lectin-like oxidized | | smooth muscle cells and endothelial cells | | Tumor therapy |
| low-density lipoprotein | | | | Myocardial ischemia |
| receptor-I | | | | Atherosclerosis |
| C-type lectin receptors: | Mannose | Immature dendritic cells, macrophages and | Unknown | Vaccine vector |
| DC-SIGN (CD209) | | megakaryocytes | | Liposome delivery |
| | | | | Adenovirus delivery |
| C-type lectin receptors: | Mannose | Immature dendritic cells, macrophages, interstitial | Yes | Vaccine vector |
| mannose receptor (CD206) | Mannan | dendritic cells, dermal dendritic cells, lymphatic | | Immunosuppression |
| | | endothelium, tracheal smooth muscle cells, kidney | | Immunoactivation |
| | | mesangial cells | | Cancer therapy |
| Fc receptors: FcγRI (CD64) | Fc | Dendritic cells, monocytes, macrophages, and | Unknown | Vaccine vector |
| | | activated neutrophils | | Tumor protection |
| | | | | Tumor therapy |

Table 1.1. Summary of dendritic cell surface receptors for antigen targeting. This table shows the expression pattern of the receptors and also indicates whether antigen targeting to the receptor requires co-stimulation for induction of immune responses.

TABLE 2

| | Groups | Day 0 | Day 12 | Day 21 | Day 24 |
|---|---|---|---|---|---|
| 1 | Control | PBS | PBS | Spleen (T cell) | IFN-γ Assay |
| | | | | Serum (Humoral) | |
| 2 | B-pVHX-6 | 500 ng | 500 ng | Spleen (T cell) | IFN-γ Assay |
| | bfFp | 20 μg | 0 | Serum (Humoral) | |
| 3 | B-pVHX-6 | 500 ng | 500 ng | Spleen (T cell) | IFN-γ Assay |
| | Anti-CD40 mAb | 25 μg | 0 | Serum (Humoral) | |
| 4 | B-pVHX-6 | 500 ng | 500 ng | Spleen (T cell) | IFN-γ Assay |
| | bfFp | 20 μg | 0 | Serum (Humoral) | |
| | Anti-CD40 mAb | 25 μg | 0 | | |
| 5 | B-OVA | 200 ng | 200 ng | Spleen (T cell) | IFN-γ Assay |
| | bfFp | 20 μg | 0 | Serum (Humoral) | |
| | Anti-CD40 mAb | 25 μg | 0 | | |
| 6 | B-MUC-1 | 200 ng | 200 ng | Spleen (T cell) | IFN-γ Assay |
| | bfFp | 20 μg | 0 | Serum (Humoral) | |
| | Anti-CD40 mAb | 25 μg | 0 | | |
| 7 | B-GM3 | 1 μg | 1 μg | Spleen (T cell) | IFN-γ Assay |
| | B-GM2 | 1 μg | 1 μg | Serum (Humoral) | |
| | bfFp | 20 μg | 0 | | |
| | Anti-CD40 mAb | 25 μg | 0 | | |
| 8 | Control | PBS | PBS | Spleen (T cell) | IFN-γ Assay |
| | | | | Serum (Humoral) | |
| 9 | B-pEBOV GP1, 2 | 500 ng | 500 ng | Spleen (T cell) | IFN-γ Assay |
| | bfFp | 20 μg | 0 | Serum (Humoral) | |
| | Anti-CD40 mAb | 25 μg | 0 | | |
| 10 | B-pSARS-CoV spike | 500 ng | 500 ng | Spleen (T cell) | IFN-γ Assay |
| | bfFp | 20 μg | 0 | Serum (Humoral) | |
| | Anti-CD40 mAb | 25 μg | 0 | | |
| 11 | B-pSARS-CoV membrane | 500 ng | 500 ng | Spleen (T cell) | IFN-γ Assay |
| | bfFp | 20 μg | 0 | Serum (Humoral) | |
| | Anti-CD40 mAb | 25 μg | 0 | | |
| 12 | Control | PBS | PBS | Spleen (T cell) | IFN-γ Assay |
| | | | | Serum (Humoral) | |
| 13 | B-OVA | 200 ng | 200 ng | Spleen (T cell) | IFN-γ Assay |
| | B-EBOV GP1 | 200 ng | 200 ng | Serum (Humoral) | |
| | B-SARS-CoV spike RBD | 200 ng | 200 ng | | |
| | B-MUC-1 | 200 ng | 200 ng | | |
| | B-Anthrax PA | 200 ng | 200 ng | | |
| | Anti-CD40 mAb | 25 μg | 0 | | |
| | Core-streptavidin | 10 μg | 0 | | |
| 14 | B-OVA | 200 ng | 200 ng | Spleen (T cell) | IFN-γ Assay |
| | B-EBOV GP1 | 200 ng | 200 ng | Serum (Humoral) | |
| | B-SARS-CoV spike RBD | 200 ng | 200 ng | | |
| | B-MUC-1 | 200 ng | 200 ng | | |
| | B-Anthrax PA | 200 ng | 200 ng | | |
| | bfFp | 20 μg | 0 | | |
| 15 | B-OVA | 200 ng | 200 ng | Spleen (T cell) | IFN-γ Assay |
| | B-EBOV GP1 | 200 ng | 200 ng | Serum (Humoral) | |
| | B-SARS-CoV spike RBD | 200 ng | 200 ng | | |
| | B-MUC-1 | 200 ng | 200 ng | | |
| | B-Anthrax PA | 200 ng | 200 ng | | |
| | bfFp | 20 μg | 0 | | |
| | Anti-CD40 mAb | 25 μg | 0 | | |

Table 2. Immunization protocol for evaluating humoral and cell-mediated immune responses to biotinylated antigens in mice (n=5 for each group). The amounts of antigens, antibodies and bfFp are either in μg or ng per mouse. All mice were injected subcutaneously near inguinal lymph node. Groups 1, 8 and 12 are the control groups and each group of the control mice were immunized and boosted subcutaneously with PBS. The first experiment demonstrated the versatility of bfFp based delivery of protein, peptide, glycolipids and DNA to dendritic cells in generating immune responses (Groups 2-7 mice). Groups 2-4 mice focused on the DNA delivery to dendritic cells and to verify the essential requirement of DC targeting vehicle and co-stimulatory molecule. Protein antigen delivery is studied in Group 5 mice, peptide delivery is focused in Group 6 mice, and glycolipids were investigated in Group 7 mice. A variety of infectious diseases viral DNA were targeted to dendritic cells using bfFp mediated delivery system (Group 9, EBOV GP1,2 DNA; Group 10, SARS-CoV spike DNA; Group 11, SARS-CoV membrane DNA). Multivalent immune responses against a mixture of antigens were studied in the third set of experiments. Groups 13-15 were designed to evaluate multivalent immune responses against proteins and peptide. All mice were boosted with the same concentration of antigen(s) in PBS 12 days following primary immunization. The mice were sacrificed after 9 days of boost.

The citation of any publication is for its disclosure prior to the filing date and should not be construed as an admission that the present invention is not entitled to antedate such publication by virtue of prior invention.

As used in this specification and the appended claims, the singular forms “a,” “an,” and “the” include plural reference unless the context clearly dictates otherwise. Unless defined otherwise all technical and scientific terms used herein have the same meaning as commonly understood to one of ordinary skill in the art to which this invention belongs.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

1. A bifunctional fusion molecule comprising:
a first functional domain comprising a first immunoglobulin variable region, a second immunoglobulin variable region and a linker for connecting the first and second variable regions;
a second functional domain comprising a moiety for binding to an antigenic agent;
wherein the first and second functional domains are linked; and wherein the first functional domain specifically binds to a surface molecule of a professional antigen-presenting cell.

2. The bifunctional fusion molecule according to claim 1, wherein the first immunoglobulin variable region is a heavy chain variable region V_{H} and the second immunoglobulin variable region is a light chain variable region V_{L}.

3. The bifunctional fusion molecule according to claim 1, wherein the moiety comprises a core streptavidin.

4. The bifunctional fusion molecule according to claim 1, wherein the antigenic agent comprises an antigen linked to a particle, wherein the particle binds to the moiety of the second functional domain of the bifunctional fusion molecule.

5. The bifunctional fusion molecule according to claim 4, wherein the antigen is a peptide, a protein, a nucleic acid molecule, or a glycolipid.

6. The bifunctional fusion molecule according to claim 4, wherein the antigen is an infectious disease antigen, a cancer antigen selected from MUC-1, GM2 and GM3, or an autoimmune disease antigen selected from transglutaminase, muscle actin, Bullous Pemphigoid antigen 1 and 2, basement membrane collagen Type IV protein, ganglioside, myelin basic protein, desmogein 3, p62/sp100/mitochondrial(M2), rheumatoid factor and topoisomerase.

7. The bifunctional fusion molecule according to claim 4, wherein the particle is biotin.

8. The bifunctional fusion molecule according to claim 1, wherein the professional antigen-presenting cell is a dendritic cell.

9. The bifunctional fusion molecule according to claim 1, wherein the surface molecule is a receptor.

10. The bifunctional fusion molecule according to claim 9, wherein the receptor is DEC-205.

11. The bifunctional fusion molecule according to claim 1, wherein the molecule is monomeric.

12. An antigen delivery system comprising:
a bifunctional fusion molecule comprising:
a first functional domain comprising an immunoglobulin heavy chain variable region, an immunoglobulin light chain variable region and a linker for connecting the heavy chain variable region and the light chain variable region;
a second functional domain comprising a moiety for binding to an antigenic agent;
wherein the first and second functional domains are linked; and wherein the first functional domain binds to a surface molecule of a professional antigen-presenting cell; and
an antigenic agent comprising an antigen and a particle;
wherein the antigen is conjugated to the particle, and wherein the particle binds to the moiety of the second domain of the bifunctional fusion molecule.

13. The antigen delivery system according to claim 12, wherein the antigen is a peptide, a protein, a nucleic acid molecule, or a glycolipid.

14. The antigen delivery system according to claim 12, wherein the antigen is an infectious disease antigen, a cancer antigen selected from MUC-1, GM2 and GM3, or an autoimmune disease antigen selected from transglutaminase, muscle actin, Bullous Pemphigoid antigen 1 and 2, basement membrane collagen Type IV protein, ganglioside, myelin basic protein, desmogein 3, p62/sp100/mitochondrial(M2), rheumatoid factor and topoisomerase.

15. The antigen delivery system according to claim 12, wherein the professional antigen-presenting cell is a dendritic cell.

16. The antigen delivery system according to claim 12, wherein the surface molecule is a receptor.

17. The antigen delivery system according to claim 16, wherein the receptor is DEC-205.

18. A method of delivering an antigen to a professional antigen-presenting cell, said method comprising:
contacting the professional antigen-presenting cell with the bifunctional fusion molecule according to claim 1; and
contacting the bifunctional fusion molecule with a plurality of antigenic agents, wherein each antigenic agent of the plurality of antigenic agents comprises an antigen linked to a particle, wherein the particle binds to the moiety of the second functional domain of the bifunctional fusion molecule.

19. A method of modulating an immune response of a subject to an antigen, said method comprising:
administering to the subject the bifunctional fusion molecule according to claim 1, a plurality of antigenic agents and optionally, a co-stimulatory molecule;
wherein each antigenic agent of the plurality of antigenic agents comprises an antigen linked to a particle, and wherein the particle binds to the moiety of the second functional domain of the bifunctional fusion molecule.

20. The method according to claim 19, wherein the co-stimulatory molecule is an anti-CD40 antibody.


SEQUENCE LISTING (26 sequences)

SEQ ID NO: 1 - DNA, 49 bp, rattus sp
atcagtgaattcgggaggtggcggatcagacatccagatgacacagtct

SEQ ID NO: 2 - DNA, 45 bp, rattus sp
tggtttcgctcatgctaggtcgaccgtggatggtgggaagataga

SEQ ID NO: 3 - DNA, 33 bp, rattus sp
gttaatgtcgacgaagtgaagctggtggaatct

SEQ ID NO: 4 - DNA, 33 bp, rattus sp
tactaagcggccgcaagctgaggagactgtgac

SEQ ID NO: 5 - DNA, 49 bp, rattus sp
atcagtgaattcgggaggtggcggatcagaagtgaagctggtggaatct

SEQ ID NO: 6 - DNA, 43 bp, rattus sp
tggtttcggtcatgataggtcgacagcagttccaggagccagt

SEQ ID NO: 7 - DNA, 33 bp, rattus sp
gttagggtcgacgacatccagatgacacagtct

SEQ ID NO: 8 - DNA, 34 bp, rattus sp
tactaagcggccgcaagcccgtttcaattccagc

SEQ ID NO: 9 - DNA, 49 bp, Streptomyces avidinii
tactaatgcggccgcggaggtggcggatcagaggccggcatcaccggca

SEQ ID NO: 10 - DNA, 31 bp, Streptomyces avidinii
attactctcgagggaggcggcggacggcttc

SEQ ID NO: 11 - DNA, 47 bp, SARS coronavirus
aagagggggatcctaccatgggtagtgaccttgaccggtgcaccact

SEQ ID NO: 12 - DNA, 49 bp, SARS coronavirus
ctcgctcgagagaattctattatgtgtaatgtaatttgacacccttgag

SEQ ID NO: 13 - DNA, 59 bp, SARS coronavirus
aagagggtcttcatatgggggatcctaccatggcagacaacggtactattaccgttgag

SEQ ID NO: 14 - DNA, 64 bp, SARS coronavirus
ctcgctcgagagaattctagtgatgatggtggtgatgctgtactagcaaagcaatattgtcgtt

SEQ ID NO: 15 - DNA, 47 bp, ebola virus
aagagggggatcctaccatgggcgttacaggaatattgcagttacct

SEQ ID NO: 16 - DNA, 49 bp, ebola virus
ctcgctcgagagaattctaaaagacaaatttgcatatacagaataaagc

SEQ ID NO: 17 - protein, 124 aa, rattus sp
EVKLVESGGGLVQPGGSLRLSCAASGFTFNDFYMNWIRQPPGQAPEWLGVIRNKGNGYTTEVNTSVKGRFTISRDNTQNILYLQMNSLRAEDTAIYYCARGGPYYYSGDDAPYWGQGVMVTVSS

SEQ ID NO: 18 - DNA, 372 bp, rattus sp
gaagtgaagctggtggaatctggaggaggtttggtacagccggggggttctctgagactctcctgtgcagcttctggattcaccttcaatgatttctacatgaactggatccgccagcctccagggcaggcacctgagtggttgggtgttattagaaacaaaggtaatggttacacaacagaggtcaatacatctgtgaaggggcggttcaccatctccagagataatacccaaaacatcctctatcttcaaatgaacagcctgagagctgaggacaccgccatttactactgtgcaagaggcggtccttattactacagtggtgacgacgccccttactggggccaaggagtcatggtcacagtctcctca

SEQ ID NO: 19 - protein, 107 aa, rattus sp
DIQMTQSPSFLSTSLGNSITITCHASQNIKGWLAWYQQKSGNAPQLLIYKASSLQSGVPSRFSGSGSGTDYIFTISNLQPEDIATYYCQHYQSFPWTFGGGTKLELK

SEQ ID NO: 20 - DNA, 321 bp, rattus sp
gacatccagatgacacagtctccgtcatttctgtctacatctcttggaaacagcatcaccatcatttgccatgccagtcagaacatcaagggttggttagcctggtaccaacaaaagtcagggaatgctcctcaactgttgatttataaggcatctagcctgcaatcaggggttccatcaagattcagtggcagtggatctggaacagattatattttcactatcagcaacctacagcctgaagatattgccacttattactgtcagcattatcaaagctttccgtggacgttcggtggaggcaccaagctggaattgaaa

SEQ ID NO: 21 - DNA, 663 bp, rattus sp
  CDS (1)..(663)
gaagtgaagctggtggaatctggaggaggtttggtacagccggggggttctctgagactctcctgtgcagcttctggattcaccttcaatgatttctacatgaactggatccgccagcctccagggcaggcacctgagtggttgggtgttattagaaacaaaggtaatggttacacaacagaggtcaatacatctgtgaaggggcggttcaccatctccagagataatacccaaaacatcctctatcttcaaatgaacagcctgagagctgaggacaccgccatttactactgtgcaagaggcggtccttattactacagtggtgacgacgccccttactggggccaaggagtcatggtcacagtctcctcagctgaaacaacagccccatctgtctatccactggctcctggaactgctctcaaaagtaactccatggtgaccctgggatgcctggtcaagggctatttccctgagccagtcaccgtgacctggaactctggagccctgtccagcggtgtgcacaccttcccagctgtcctgcagtctggactctacactctcaccagctcagtgactgtaccctccagcacctggtccagccaggccgtcacctgcaacgtagcccacccggccagcagcaccaaggtggacaagaaaatt

SEQ ID NO: 22 - protein, 221 aa, rattus sp
EVKLVESGGGLVQPGGSLRLSCAASGFTFNDFYMNWIRQPPGQAPEWLGVIRNKGNGYTTEVNTSVKGRFTISRDNTQNILYLQMNSLRAEDTAIYYCARGGPYYYSGDDAPYWGQGVMVTVSSAETTAPSVYPLAPGTALKSNSMVTLGCLVKGYFPEPVTVTWNSGALSSGVHTFPAVLQSGLYTLTSSVTVPSSTWSSQAVTCNVAHPASSTKVDKKI

SEQ ID NO: 23 - DNA, 663 bp, rattus sp
aattttcttgtccaccttggtgctgctggccgggtgggctacgttgcaggtgacggcctggctggaccaggtgctggagggtacagtcactgagctggtgagagtgtagagtccagactgcaggacagctgggaaggtgtgcacaccgctggacagggctccagagttccaggtcacggtgactggctcagggaaatagcccttgaccaggcatcccagggtcaccatggagttacttttgagagcagttccaggagccagtggatagacagatggggctgttgtttcagctgaggagactgtgaccatgactccttggccccagtaaggggcgtcgtcaccactgtagtaataaggaccgcctcttgcacagtagtaaatggcggtgtcctcagctctcaggctgttcatttgaagatagaggatgttttgggtattatctctggagatggtgaaccgccccttcacagatgtattgacctctgttgtgtaaccattacctttgtttctaataacacccaaccactcaggtgcctgccctggaggctggcggatccagttcatgtagaaatcattgaaggtgaatccagaagctgcacaggagagtctcagagaaccccccggctgtaccaaacctcctccagattccaccagcttcacttc

SEQ ID NO: 24 - DNA, 643 bp, rattus sp
  CDS (1)..(642)
gacatccagatgacacagtctccgtcatttctgtctacatctcttggaaacagcatcaccatcatttgccatgccagtcagaacatcaagggttggttagcctggtaccaacaaaagtcagggaatgctcctcaactgttgatttataaggcatctagcctgcaatcaggggttccatcaagattcagtggcagtggatctggaacagattatattttcactatcagcaacctacagcctgaagatattgccacttattactgtcagcattatcaaagctttccgtggacgttcggtggaggcaccaagctggaattgaaacgggctgatgctgcaccaactgtatctatcttcccaccatccacggaacagttagcaactggaggtgcctcagtcgtgtgcctcatgaacaacttctatcccagagacatcagtgtcaagtggaagattgatggcactgaacgacgagatggtgtcctggacagtgttactgatcaggacagcaaagacagcacgtacagcatgagcagcaccctctcgttgaccaaggctgactatgaaagtcataacctctatacctgtgaggttgttcatatgacatcatcctcacccgtcgtcaagagcttcaacaggaatgagtgtt

SEQ ID NO: 25 - protein, 214 aa, rattus sp
DIQMTQSPSFLSTSLGNSITIICHASQNIKGWLAWYQQKSGNAPQLLIYKASSLQSGVPSRFSGSGSGTDYIFTISNLQPEDIATYYCQHYQSFPWTFGGGTKLELKRADAAPTVSIFPPSTEQLATGGASVVCLMNNFYPRDISVKWKIDGTERRDGVLDSVTDQDSKDSTYSMSSTLSLTKADYESHNLYTCEVVHMTSSSPVVKSFNRNEC

SEQ ID NO: 26 - DNA, 643 bp, rattus sp
aacactcattcctgttgaagctcttgacgacgggtgaggatgatgtcatatgaacaacctcacaggtatagaggttatgactttcatagtcagccttggtcaacgagagggtgctgctcatgctgtacgtgctgtctttgctgtcctgatcagtaacactgtccaggacaccatctcgtcgttcagtgccatcaatcttccacttgacactgatgtctctgggatagaagttgttcatgaggcacacgactgaggcacctccagttgctaactgttccgtggatggtgggaagatagatacagttggtgcagcatcagcccgtttcaattccagcttggtgcctccaccgaacgtccacggaaagctttgataatgctgacagtaataagtggcaatatcttcaggctgtaggttgctgatagtgaaaatataatctgttccagatccactgccactgaatcttgatggaacccctgattgcaggctagatgccttataaatcaacagttgaggagcattccctgacttttgttggtaccaggctaaccaacccttgatgttctgactggcatggcaaatgatggtgatgctgtttccaagagatgtagacagaaatgacggagactgtgtcatctggatgtc